(19) United States
(12) Patent Application Publication
(10) Pub. No.: US 2021/0241855 A1
(43) Pub. Date: Aug. 5, 2021

(54) METHODS AND TEST KITS FOR DETERMINING MALE FERTILITY STATUS

(71) Applicant: ANDROVIA LIFESCIENCES, LLC, Mountainside, NJ (US)

(72) Inventors: Alexander J. TRAVIS, Ithaca, NY (US); John D. COOK, Houston, TX (US)

(73) Assignee: ANDROVIA LIFESCIENCES, LLC, Mountainside, NJ (US)

(21) Appl. No.: 17/051,912

(22) PCT Filed: May 2, 2019
(86) PCT No.: PCT/US2019/030372

§ 371 Date: Oct. 30, 2020

Related U.S. Application Data
(60) Provisional application No. 62/665,870, filed on May 2, 2018.

(51) Int. Cl. G16B 40/00; G01N 1/30; G01N 33/569; G16B 5/20; G16H 10/40; G16H 50/70; G16H 50/30

(52) U.S. Cl. CPC G16B 40/00; G01N 1/30; G01N 33/56966; G01N 2800/367; G16H 10/40; G16H 50/70; G16H 50/30; G16B 5/20

(57) ABSTRACT

This disclosure provides a method for determining male fertility status, and its relationship to the probability of generating a pregnancy as calculated using a regression model. The method comprises determining G_{M1} localization patterns following induced sperm capacitation, identifying the percentage of various patterns, particularly the ratio of [(AA+APM)/total number of G_{M1} localization patterns] and determining if the percentage of certain GMI localization patterns in response to induced capacitation is altered. Based on the change in the percentage of localization patterns of certain patterns in response to induced capacitation, alone or in combination with other sperm attributes, male fertility status can be identified.

### FIELD OF THE DISCLOSURE

This invention relates generally to the field of male fertility and more specifically to determining male fertility status based on GM₁ ganglioside distribution patterns following induced sperm capacitation.

### BACKGROUND OF THE DISCLOSURE

In the US, 10% of couples have medical appointments related to infertility with 40% of infertility being associated with the male. Globally, this translates to over 73 million infertile couples. Typical male reproductive health exams assess sperm number, appearance, and motility. Unfortunately, half of infertile men have sperm that meet normal parameters for these descriptive criteria and are only identified as having “idiopathic infertility” after repeatedly failing at both natural conception and techniques of assisted reproduction such as intra-uterine insemination (IUI). Because each failed cycle inflicts great physical, emotional, and financial tolls on couples and it costs the US healthcare system over $5 billion annually, there is a tremendous need for a practical test of sperm function. Data on sperm function would allow clinicians to direct their patients toward a technology of assisted reproduction that would give them the best chance to conceive.

Upon entrance into the female tract, sperm are not immediately able to fertilize an egg. Rather, they must undergo a process of functional maturation known as “capacitation.” This process relies upon their ability to respond to specific stimuli by having specific changes in their cell membrane, namely a change in the distribution pattern of the ganglioside G_{M1} in response to exposure to stimuli for capacitation.

Various G_{M1} localization patterns have been identified and associated with capacitation or non-capacitation. In particular, apical acrosome (AA) G_{M1} localization patterns and acrosomal plasma membrane (APM) G_{M1} localization patterns have been associated with capacitation in bovine and human sperm. Sperm capacitation can be quantitatively expressed as a Cap-Score™ value, generated via the Cap-Score™ Sperm Function Test or Cap-Score™ Male Fertility Assay (“Cap-Score™ Test” or “Cap-Score™”), which is defined as ([number of apical acrosome (AA) G_{M1} localization patterns+number of acrosomal plasma membrane (APM) G_{M1} localization patterns]/total number of G_{M1} labeled localization patterns) where the number of each localization pattern is measured and then ultimately converted to a percentage score. In addition to APM G_{M1} localization patterns and AA G_{M1} localization patterns, the other labeled localization patterns included Lined-Cell G_{M1} localization patterns, intermediate (INTER) G_{M1} localization patterns, post acrosomal plasma membrane (PAPM) G_{M1} localization patterns, apical acrosome/post acrosome (AA/PA) G_{M1} localization patterns, equatorial segment (ES) G_{M1} localization patterns, and diffuse (DIFF) G_{M1} localization patterns. (Travis et al., “Impacts of common semen handling methods on sperm function,” The Journal of Urology, 195 (4), e909 (2016)).

### SUMMARY OF THE INVENTION

In an embodiment of the invention, disclosed herein are methods and kits for determining male fertility status. In one embodiment, this disclosure describes a method for identifying male fertility status based on a change in the number of certain G_{M1} localization patterns in response to at least one capacitation stimulus.

An embodiment disclosed herein is a method for determining male fertility status. In one embodiment, the method comprising the following steps: A sample of sperm cells exposed to capacitation stimuli is treated with a fluorescence label. One or more fluorescence images of such sperm cells is obtained wherein the images display one or more G_{M1} localization patterns. Sperm cells expressing an apical acrosome (AA) G_{M1} localization pattern and an acrosomal plasma membrane (APM) G_{M1} localization pattern are each assigned to a capacitated state and all other fluorescence-labeled G_{M1} localization patterns are assigned to a non-capacitated state. The non-capacitated G_{M1} localization patterns include INTER, PAPM (Post Acrosomal Plasma Membrane), AA/PA (Apical Acrosome/Post Acrosome), ES (Equatorial Segment) DIFF (Diffuse), and Lined-Cell. The percentage of [(AA G_{M1} localization patterns plus APM G_{M1} localization patterns)/total G_{M1} localization patterns] is calculated. In one embodiment, a fertility threshold associated with a percentage of [(AA G_{M1} localization patterns plus APM G_{M1} localization patterns)/total G_{M1} localization patterns] is determined, wherein a reference percentage of [(AA G_{M1} localization patterns plus APM G_{M1} localization patterns)/total G_{M1} localization patterns] is based on distribution statistics of a known fertile population corresponding to: greater than a percentage than one standard deviation below the reference mean percentage indicates fertile; less than a percentage that is one standard deviation below the reference mean percentage and greater than a percentage that is two standard deviations below the reference mean percentage indicates sub-fertile; less than a percentage that is two standard deviations below the reference mean percentage indicates infertile. In an embodiment, the percentage of [(AA G_{M1} localization patterns plus APM G_{M1} localization patterns)/total G_{M1} localization patterns] for the labeled, fixed in vitro capacitated sperm sample is compared to the reference percentage of [(AA G_{M1} localization patterns plus APM G_{M1} localization patterns)/total G_{M1} localization patterns]. The fertility threshold is identified based on the comparison.

In another embodiment, a fertility threshold associated with a percentage of [(AA G_{M1} localization patterns plus APM G_{M1} localization patterns)/total G_{M1} localization patterns] is determined, wherein a reference percentage of [(AA G_{M1} localization patterns plus APM G_{M1} localization patterns)/total G_{M1} localization patterns] is based on distribution statistics of a known fertile population corresponding to: greater than a percentage that is one standard deviation below the reference mean percentage indicates normal male fertility; less than a percentage that is one standard deviation below the reference mean percentage indicates abnormal male fertility. The percentage of [(AA G_{M1} localization patterns plus APM G_{M1} localization patterns)/total G_{M1} localization patterns] for the labeled, fixed capacitated sperm sample is compared to the reference percentage of [(AA G_{M1} localization patterns plus APM G_{M1} localization patterns)/total G_{M1} localization patterns]. The fertility threshold is identified based on the comparison.

Another embodiment disclosed herein is a method for determining male fertility status. In one embodiment, the method includes exposing a first portion of a sperm sample from a male to non-capacitating conditions to obtain an in vitro non-capacitated sperm sample; exposing a second portion of the sperm sample to capacitating conditions to obtain an in vitro capacitated sperm sample; fixing the in vitro non-capacitated sperm sample and the in vitro capacitated sperm sample with a fixative for a time period of at least: one hour, two hours, three hours, four hours, five hours, six, hours, seven hours, eight hours, nine hours, ten hours, eleven hours, twelve hours, eighteen hours or twenty four hours; treating the fixed in vitro non-capacitated sperm sample and the fixed in vitro capacitated sperm sample with a labeling molecule for G_{M1} localization patterns, wherein the labeling molecule has a detectable label; identifying more than one labeled G_{M1} localization patterns for the labeled fixed in vitro non-capacitated sperm sample and the labeled fixed in vitro capacitated sperm sample, said G_{M1} labeled localization patterns being an apical acrosome (AA) G_{M1} localization pattern, an acrosomal plasma membrane (APM) G_{M1} localization pattern, a Lined-Cell G_{M1} localization pattern and all other labeled G_{M1} localization patterns; comparing the labeled G_{M1} localization patterns for the labeled fixed in vitro non-capacitated sperm sample to the labeled G_{M1} localization patterns for the labeled fixed in vitro capacitated sperm sample; based on the comparison, assigning the apical acrosome (AA) G_{M1} localization pattern and the acrosomal plasma membrane (APM) G_{M1} localization pattern to a capacitated state and assigning the Lined-Cell G_{M1} localization pattern and all other labeled G_{M1} localization patterns to a non-capacitated state; and characterizing a fertility status of the male based on the identified G_{M1} labeled localization patterns for the labeled fixed in vitro non-capacitated sperm sample and the labeled fixed in vitro capacitated sperm sample. In one embodiment, the characterizing step comprises the steps of: determining a fertility threshold associated with a percentage of [(AA G_{M1} localization patterns plus APM G_{M1} localization patterns)/total G_{M1} localization patterns] for the labeled fixed in vitro capacitated sperm sample; wherein a reference percentage of [(AA G_{M1} localization patterns plus APM G_{M1} localization patterns)/total G_{M1} localization patterns], based on distribution statistics of a known fertile population corresponding to: greater than a percentage that is one standard deviation below the reference mean percentage indicates fertile; less than a percentage that is one standard deviation below the reference mean percentage and greater than a percentage that is two standard deviations below the reference mean percentage indicates sub-fertile; greater than two standard deviations below the reference mean percentage indicates infertile. The percentage of [(AA G_{M1} localization patterns plus APM G_{M1} localization patterns)/total G_{M1} localization patterns] for the labeled fixed in vitro capacitated sperm sample is compared to the reference percentage of [(AA G_{M1} localization patterns plus APM G_{M1} localization patterns)/total G_{M1} localization patterns]. The fertility threshold is identified based on the comparison.

In another embodiment, a fertility threshold associated with a percentage of [(AA G_{M1} localization patterns plus APM G_{M1} localization patterns)/total G_{M1} localization patterns] is determined, wherein a reference percentage of [(AA G_{M1} localization patterns plus APM G_{M1} localization patterns)/total G_{M1} localization patterns] is based on distribution statistics of a known fertile population corresponding to: greater than a percentage that is one standard deviation below the reference mean percentage indicates normal male fertility; less than a percentage that is one standard deviation below the reference mean percentage indicates abnormal male fertility. The percentage of [(AA G_{M1} localization patterns plus APM G_{M1} localization patterns)/total G_{M1} localization patterns] for the labeled fixed in vitro capacitated sperm sample is compared to the reference percentage of [(AA G_{M1} localization patterns plus APM G_{M1} localization patterns)/total G_{M1} localization patterns]. The fertility threshold is identified based on the comparison.

In one embodiment, prior to the exposing steps, a semen sample is treated to decrease semen viscosity using a wide orifice pipette made of non-metallic material and using a reagent that does not damage sperm membranes chosen from the various reagents that are used to decrease semen viscosity. In some such embodiments, the membrane damaging reagents potentially may include (i) a protease; (ii) a nuclease (iii) a mucolytic agent; (iv) a lipase; (v) an esterase and (vi) glycoside hydrolases. In some embodiments, the identifying step is repeated until the number of Lined-Cell G_{M1} localization patterns is substantially constant. In one such embodiment, after the identifying step is performed, determining the number of Lined-Cell G_{M1} localization patterns, for the labeled fixed in vitro capacitated sperm until the number is less than 5%, less than 3% of the total number of labeled cells; or ranges from 1% to 5%, 2 to 5% of the total number of labeled cells. In another such embodiment, after the identifying step is performed, the number of Lined-Cell G_{M1} localization patterns, for the labeled fixed in vitro non-capacitated sperm is determined until the number is less than: 25%, 20%, 15% or 10% of the total number of labeled cells; or ranges from 2% to 25%; 2% to 20%; 2 to 15%; 2 to 10%; 2 to 5% of the total number of labeled cells. In some embodiments the wide orifice pipette has a gauge size of at least 18 gauge, 16 gauge or 14 gauge. In some embodiments, the wide orifice pipette has an orifice size of at least 1 mm, 1.2 mm or 1.4 mm.

In another such embodiment, the characterizing step further includes the steps of: determining the number of each G_{M1} labeled localization patterns for a predetermined number of the labeled fixed in vitro non-capacitated sperm sample; determining the number of each G_{M1} labeled localization patterns for a predetermined number of the labeled fixed in vitro capacitated sperm sample; calculating a ratio for a sum of the number of AA G_{M1} localization patterns and number of APM G_{M1} localization patterns over a sum of the total number of G_{M1} labeled localization patterns for the labeled fixed in vitro non-capacitated sperm sample; and calculating a ratio for a sum of the number of AA G_{M1} localization patterns and number of APM G_{M1} localization patterns over a sum of the total number of G_{M1} labeled localization patterns for the labeled fixed in vitro capacitated sperm sample.

In one embodiment disclosed herein the method further includes the steps of: comparing the ratio for the labeled fixed in vitro non-capacitated sperm to a ratio of labeled fixed in vitro non-capacitated sperm having a known fertility status; and comparing the ratio for the labeled fixed in vitro capacitated sperm to a ratio of labeled fixed in vitro capacitated sperm having a known fertility status.

Yet another embodiment disclosed herein is a method for determining male fertility status. In one embodiment, the method includes the steps of: obtaining a first portion of a sperm sample from a male that has been exposed to in vitro non-capacitating conditions, fixed in a fixative for at least: one hour, two hours, four hours, eight hours, twelve hours, eighteen hours or twenty four hours, and treated with a labeling molecule for G_{M1} localization patterns, wherein the labeling molecule has a detectable label; obtaining a second portion of the sperm sample that has been exposed to in vitro capacitating conditions, fixed in a fixative, and treated with the labeling molecule for G_{M1} localization patterns; identifying more than one G_{M1} labeled localization patterns for the labeled fixed in vitro non-capacitated sperm sample and the labeled fixed in vitro capacitated sperm sample, said G_{M1} labeled localization patterns being an apical acrosome (AA) G_{M1} localization pattern, an acrosomal plasma membrane (APM) G_{M1} localization pattern, a Lined-Cell G_{M1} localization pattern and all other labeled G_{M1} localization patterns; comparing the labeled G_{M1} localization patterns for the labeled fixed in vitro non-capacitated sperm sample to the labeled G_{M1} localization patterns for the labeled fixed in vitro capacitated sperm sample; based on the comparison, assigning the apical acrosome (AA) G_{M1} localization pattern and the acrosomal plasma membrane (APM) G_{M1} localization pattern to a capacitated state and assigning the Lined-Cell G_{M1} localization pattern and all other labeled G_{M1} localization patterns to a non-capacitated state; and characterizing a fertility status of the male based on the identified G_{M1} labeled localization patterns for the labeled fixed in vitro non-capacitated sperm sample and the labeled fixed in vitro capacitated sperm sample. In one such embodiment, the characterizing step comprises the steps of: determining a fertility threshold associated with a percentage of [(AA G_{M1} localization patterns plus APM G_{M1} localization patterns)/total G_{M1} localization patterns] for the labeled fixed in vitro capacitated sperm sample; wherein a reference percentage of [(AA G_{M1} localization patterns plus APM G_{M1} localization patterns)/total G_{M1} localization patterns], based on distribution statistics of a known fertile population corresponding to: greater than a percentage that is one standard deviation below the reference mean percentage indicates fertile; less than a percentage that is one standard deviation below the reference mean percentage and greater than a percentage that is two standard deviations below the reference mean percentage indicates sub-fertile; less than a percentage that is two standard deviations below the reference mean percentage indicates infertile; comparing the percentage of [(AA G_{M1} localization patterns plus APM G_{M1} localization patterns)/total G_{M1} localization patterns] for the labeled fixed in vitro capacitated sperm sample to the reference percentage of [(AA G_{M1} localization patterns plus APM G_{M1} localization patterns)/total G_{M1} localization patterns] and identifying the fertility threshold based on the comparison.

In another embodiment, a fertility threshold associated with a percentage of [(AA G_{M1} localization patterns plus APM G_{M1} localization patterns)/total G_{M1} localization patterns] is determined, wherein a reference percentage of [(AA G_{M1} localization patterns plus APM G_{M1} localization patterns)/total G_{M1} localization patterns], based on distribution statistics of a known fertile population corresponding to: greater than a percentage that is one standard deviation below the reference mean percentage indicates normal male fertility; less than a percentage that is one standard deviation below the reference mean percentage indicates abnormal fertility. The percentage of [(AA G_{M1} localization patterns plus APM G_{M1} localization patterns)/total G_{M1} localization patterns] for the labeled fixed in vitro capacitated sperm sample is compared to the reference percentage of [(AA G_{M1} localization patterns plus APM G_{M1} localization patterns)/total G_{M1} localization patterns]. The fertility threshold is identified based on the comparison.

In some embodiments, the identifying step is repeated until the number of Lined-Cell G_{M1} localization patterns is substantially constant. In one such embodiment, after the identifying step is performed, determining the number of Lined-Cell G_{M1} localization patterns, for the labeled fixed in vitro capacitated sperm until the number is less than 5%, less than 3% of the total number of labeled cells; or ranges from 1% to 5%, 2 to 5% of the total number of labeled cells. In another such embodiment, after the identifying step is performed, determining the number of Lined-Cell G_{M1} localization patterns, for the labeled fixed in vitro non-capacitated sperm until the number is less than: 25%, 20%, 15% or 10% of the total number of labeled cells; or ranges from 2% to 25%; 2% to 20%; 2 to 15%; 2 to 10%; 2 to 5% of the total number of labeled cells.

In one embodiment of such method, the method further includes the steps of: determining the number of each G_{M1} labeled localization patterns for a predetermined number of the labeled fixed in vitro non-capacitated sperm sample and the labeled fixed in vitro capacitated sperm sample, and calculating a ratio for a sum of the number of AA G_{M1} localization patterns and number of APM G_{M1} localization patterns over a sum of the total number of G_{M1} localization patterns each for the labeled fixed in vitro non-capacitated sperm sample and the labeled fixed in vitro capacitated sperm sample.

In another embodiment, the characterizing step further includes the steps of: comparing the ratio for the labeled fixed in vitro capacitated sperm sample to ratios of G_{M1} localization patterns of in vitro capacitated sperm for males having a known fertility status; and comparing the ratio for the labeled fixed in vitro non-capacitated sperm sample to ratios of G_{M1} localization patterns in vitro non-capacitated sperm for males having a known fertility status.

Still yet another embodiment disclosed herein is a method for determining male fertility status. In one embodiment, the method includes the steps of: exposing, in vitro, a sperm sample from a male to capacitating conditions; fixing the capacitated sperm sample with a fixative for at least: one hour, two hours, three hours, four hours, five hours, six hours, seven hours, eight hours, nine hours, ten hours, eleven hours, twelve hours, eighteen hours or twenty four hours; treating the fixed in vitro capacitated sperm sample with a labeling molecule for G_{M1} localization patterns, wherein the labeling molecule has a detectable label; identifying more than one G_{M1} labeled localization patterns for the labeled fixed in vitro capacitated sperm sample, said G_{M1} labeled localization patterns being an apical acrosome (AA) G_{M1} localization pattern, an acrosomal plasma membrane (APM) G_{M1} localization pattern, a Lined-Cell G_{M1} localization pattern and all other labeled G_{M1} localization patterns; assigning the apical acrosome (AA) G_{M1} localization pattern and the acrosomal plasma membrane (APM) G_{M1} localization pattern to a capacitated state and assigning the Lined-Cell G_{M1} localization pattern and all other labeled G_{M1} localization patterns to a non-capacitated state; and characterizing a fertility status of the male. In one embodiment, the characterizing step comprises the steps of: determining a fertility threshold associated with a percentage of [(AA G_{M1} localization patterns plus APM G_{M1} localization patterns)/total G_{M1} localization patterns] for the labeled fixed in vitro capacitated sperm sample; wherein a reference percentage of [(AA G_{M1} localization patterns plus APM G_{M1} localization patterns)/total G_{M1} localization patterns], based on distribution statistics of a known fertile population corresponding to: greater than a percentage that is one standard deviation below the reference mean percentage indicates fertile; less than a percentage that is one standard deviation below the reference mean percentage and greater than a percentage that is two standard deviations below the reference mean percentage indicates sub-fertile; less than two standard deviations below the reference mean percentage indicates infertile; comparing the percentage of [(AA G_{M1} localization patterns plus APM G_{M1} localization patterns)/total G_{M1} localization patterns] for the labeled fixed in vitro capacitated sperm sample to the reference percentage of [(AA G_{M1} localization patterns plus APM G_{M1} localization patterns)/total G_{M1} localization patterns] and identifying the fertility threshold based on the comparison.

In another embodiment, a fertility threshold associated with a percentage of [(AA G_{M1} localization patterns plus APM G_{M1} localization patterns)/total G_{M1} localization patterns] is determined, wherein a reference percentage of [(AA G_{M1} localization patterns plus APM G_{M1} localization patterns)/total G_{M1} localization patterns], based on distribution statistics of a known fertile population corresponding to: greater than a percentage that is one standard deviation below the reference mean percentage indicates normal male fertility; less than a percentage that is one standard deviation below the reference mean percentage indicates abnormal male fertility. The percentage of [(AA G_{M1} localization patterns plus APM G_{M1} localization patterns)/total G_{M1} localization patterns] for the labeled fixed in vitro capacitated sperm sample is compared to the reference percentage of [(AA G_{M1} localization patterns plus APM G_{M1} localization patterns)/total G_{M1} localization patterns]. The fertility threshold is identified based on the comparison.

In one embodiment, prior to the exposing steps, a semen sample is treated to decrease semen viscosity using a wide orifice pipette made of non-metallic material and using a reagent that does not damage sperm membrane chosen from the various reagents that are used to decrease semen viscosity. In some embodiments, the membrane damaging reagent may be potentially selected from the group consisting of (i) a protease; (ii) a nuclease (iii) a mucolytic agent; (iv) a lipase; (v) an esterase and (vi) glycoside hydrolases. In some embodiments, the identifying step is repeated until the number of Lined-Cell G_{M1} localization patterns is substantially constant. In one such embodiment, after the identifying step is performed, determining the number of Lined-Cell G_{M1} localization patterns, for the labeled fixed in vitro capacitated sperm until the number is less than 5%, less than 3% of the total number of labeled cells; or ranges from 1% to 5%, 2 to 5% of the total number of labeled cells. In another such embodiment, after the identifying step is performed, determining the number of Lined-Cell G_{M1} localization patterns, for the labeled fixed in vitro non-capacitated sperm until the number is less than: 25%, 20%, 15% or 10% of the total number of labeled cells; or ranges from 2% to 25%; 2% to 20%; 2 to 15%; 2 to 10%; 2 to 5% of the total number of labeled cells. In some embodiments the wide orifice pipette has a gauge size of at least 18 gauge, 16 gauge or 14 gauge. In some embodiments, the wide orifice pipette has an orifice size of at least 1 mm, 1.2 mm or 1.4 mm.

In one embodiment of such method, the method further includes the steps of: comparing the ratio of G_{M1} localization patterns to ratios of G_{M1} localization patterns for males having a known fertility status. In one such embodiment, the comparing step includes the steps of: determining the number of each G_{M1} labeled localization patterns for a predetermined number of the labeled fixed in vitro capacitated sperm sample, and calculating a ratio for a sum of the number of AA G_{M1} localization patterns and number of APM G_{M1} localization patterns over a sum of the total number of G_{M1} labeled localization patterns.

Another embodiment disclosed herein is a method for determining male fertility status. In one embodiment, the method includes the steps of: obtaining a first portion of a sperm sample from a male that has been exposed to in vitro capacitating conditions, fixed in a fixative for at least: one hour, two hours, four hours, eight hours, twelve hours, eighteen hours or twenty four hours, and stained with a labeling molecule for G_{M1} localization patterns, wherein the labeling molecule has a detectable label; identifying more than one G_{M1} labeled localization patterns for the labeled fixed in vitro capacitated sperm sample, said G_{M1} localization patterns being an apical acrosome (AA) G_{M1} localization pattern, an acrosomal plasma membrane (APM) G_{M1} localization pattern, a Lined-Cell G_{M1} localization pattern and all other labeled G_{M1} localization patterns; assigning the apical acrosome (AA) G_{M1} localization pattern and the acrosomal plasma membrane (APM) G_{M1} localization pattern to a capacitated state and assigning the Lined-Cell G_{M1} localization pattern and all other labeled G_{M1} localization patterns to a non-capacitated state; and characterizing a fertility status of the male. In some embodiments, characterizing step comprises the steps of: determining a fertility threshold associated with a percentage of [(AA G_{M1} localization patterns plus APM G_{M1} localization patterns)/total G_{M1} localization patterns] for the labeled fixed in vitro capacitated sperm sample; wherein a reference percentage of [(AA G_{M1} localization patterns plus APM G_{M1} localization patterns)/total G_{M1} localization patterns], based on distribution statistics of a known fertile population corresponding to: greater than a percentage that is one standard deviation below the reference mean percentage indicates fertile; less than a percentage that is one standard deviation below the reference mean percentage and greater than a percentage that is two standard deviations below the reference mean percentage indicates sub-fertile; less than a percentage that is two standard deviations below the reference mean percentage indicates infertile; comparing the percentage of [(AA G_{M1} localization patterns plus APM G_{M1} localization patterns)/total G_{M1} localization patterns] for the labeled fixed in vitro capacitated sperm sample to the reference percentage of [(AA G_{M1} localization patterns plus APM G_{M1} localization patterns)/total G_{M1} localization patterns] and identifying the fertility threshold based on the comparison.

In another embodiment, a fertility threshold associated with a percentage of [(AA G_{M1} localization patterns plus APM G_{M1} localization patterns)/total G_{M1} localization patterns] is determined, wherein a reference percentage of [(AA G_{M1} localization patterns plus APM G_{M1} localization patterns)/total G_{M1} localization patterns], based on distribution statistics of a known fertile population corresponding to: greater than a percentage that is one standard deviation below the reference mean percentage indicates normal male fertility; less than a percentage that is one standard deviation below the reference mean percentage indicates abnormal male fertility. The percentage of [(AA G_{M1} localization patterns plus APM G_{M1} localization patterns)/total G_{M1} localization patterns] for the labeled fixed in vitro capacitated sperm sample is compared to the reference percentage of [(AA G_{M1} localization patterns plus APM G_{M1} localization patterns)/total G_{M1} localization patterns]. The fertility threshold is identified based on the comparison.

In some embodiments, the identifying step is repeated until the number of Lined-Cell G_{M1} localization patterns is substantially constant. In one such embodiment, after the identifying step is performed, determining the number of Lined-Cell G_{M1} localization patterns, for the labeled fixed in vitro capacitated sperm until the number is less than 5%, less than 3% of the total number of labeled cells; or ranges from 1% to 5%, 2 to 5% of the total number of labeled cells. In another such embodiment, after the identifying step is performed, determining the number of Lined-Cell G_{M1} localization patterns, for the labeled fixed in vitro non-capacitated sperm until the number is less than: 25%, 20%, 15% or 10% of the total number of labeled cells; or ranges from 2% to 25%; 2% to 20%; 2 to 15%; 2 to 10%; 2 to 5% of the total number of labeled cells.

In one embodiment of such method, the method further includes the steps of: comparing the ratio of G_{M1} localization patterns to ratios of G_{M1} localization patterns for males having a known fertility status. In one such embodiment, the comparing step includes the steps of: determining the number of each G_{M1} labeled localization patterns for a predetermined number of the labeled fixed in vitro capacitated sperm sample, and calculating a ratio for a sum of the number of AA G_{M1} localization patterns and number of APM G_{M1} localization patterns over a sum of the total number of G_{M1} labeled localization patterns.

Another embodiment disclosed herein is a method for determining male fertility status. In one embodiment, the method includes the steps of: obtaining a sperm sample, wherein at least a portion of the sperm sample has been exposed to in vitro capacitating conditions to obtain in vitro capacitated sperm, has been exposed to a fixative for at least: one hour, two hours, four hours, eight hours, twelve hours, eighteen hours or twenty four hours, and has been stained for G_{M1}; obtaining values for one or more semen parameters of the sperm sample; determining a Cap-Score of the labeled fixed in vitro capacitated sperm sample based on one or more G_{M1} labeled localization patterns, said G_{M1} labeled localization patterns being an apical acrosome (AA) G_{M1} localization pattern, a post-acrosomal plasma membrane (APM) G_{M1} localization pattern, a Lined-Cell G_{M1} localization pattern and all other labeled G_{M1} localization patterns; and calculating a male fertility index (MFI) value of the male based on the determined CAP score and the one or more obtained semen parameters. In one embodiment, the one or more semen parameters of the sperm sample are selected from the group consisting of volume of the original sperm sample, concentration of sperm, motility of sperm, and morphology of sperm. In some embodiments, the identifying step is repeated until the number of Lined-Cell G_{M1} localization patterns is substantially constant.

In one such embodiment, after the identifying step is performed, determining the number of Lined-Cell G_{M1} localization patterns, for the labeled fixed in vitro capacitated sperm until the number is less than 5%, less than 3% of the total number of labeled cells; or ranges from 1% to 5%, 2 to 5% of the total number of labeled cells. In another such embodiment, after the identifying step is performed, determining the number of Lined-Cell G_{M1} localization patterns, for the labeled fixed in vitro non-capacitated sperm until the number is less than: 25%, 20%, 15% or 10% of the total number of labeled cells; or ranges from 2% to 25%; 2% to 20%; 2 to 15%; 2 to 10%; 2 to 5% of the total number of labeled cells.

In various embodiments of the methods described herein, the more than one of G_{M1} labeled localization patterns comprises AA G_{M1} localization pattern, APM G_{M1} localization pattern, Lined-Cell G_{M1} localization pattern, intermediate (INTER) G_{M1} localization pattern, post acrosomal plasma membrane (PAPM) G_{M1} localization pattern, apical acrosome/post acrosome (AA/PA) G_{M1} localization pattern, equatorial segment (ES) G_{M1} localization pattern, and diffuse (DIFF) G_{M1} localization pattern.

In one embodiment, exposing the first portion of the sperm sample to non-capacitating conditions and exposing the second portion of the sperm sample to capacitating conditions occur concurrently.

In one embodiment disclosed herein is a kit for identifying a fertility status of a male comprising: a diagram illustrating one or more G_{M1} localization patterns of capacitated sperm and one of more G_{M1} localization patterns of non-capacitated sperm, wherein said G_{M1} localization patterns of capacitated sperm and G_{M1} localization patterns of non-capacitated sperm are reflective of known fertility status; a wide orifice pipette having an orifice of sufficient size in diameter to prevent shearing of a sperm membrane; one or more of the following: capacitating media, non-capacitating media, fixative composition, labeling reagents for determining G_{M1} localization patterns; with the proviso that the fixative composition does not damage sperm membranes, wherein the capacitating media and non-capacitating media, when applied in vitro to sperm cells, produce G_{M1} localization patterns indicative of capacitated sperm and patterns indicative of non-capacitated sperm as reflected in the diagram. In one embodiment, the kit contains instructions for handling sperm in order to avoid damaging the sperm membrane. In some embodiments the wide orifice pipette has a gauge size of at least 18 gauge, 16 gauge or 14 gauge. In some embodiments, the wide orifice pipette has an orifice size of at least 1 mm, 1.2 mm or 1.4 mm.

In certain embodiments described herein, the in vitro capacitating conditions include exposure to one or more of bicarbonate ions, calcium ions, and a mediator of sterol efflux. In some embodiments, the mediator of sterol efflux is 2-hydroxy-propyl-β-cyclodextrin, methyl-β-cyclodextrin, serum albumin, high density lipoprotein, phospholipid vesicles, fetal cord serum ultrafiltrate, fatty acid binding proteins, or liposomes. In one embodiment, the mediator of sterol efflux is 2-hydroxy-propyl-β-cyclodextrin.

In one embodiment, the non-capacitating conditions include the lack of exposure to one or more of bicarbonate ions, calcium ions, and a mediator of sterol efflux.

In certain embodiments described herein, the fixative is an aldehyde fixative. In one embodiment, the fixative includes paraformaldehyde, glutaraldehyde or combinations thereof. In certain embodiments, the affinity molecule for G_{M1} is fluorescent labeled cholera toxin b subunit.

In certain embodiments described herein, the method comprises characterizing a fertility status of the male by applying one or more pre-trained fertility classifiers to data obtained from the sperm sample, wherein the data obtained from the sperm sample comprises a ratio between (i) a combination of the AA G_{M1} localization pattern and APM G_{M1} localization patterns and (ii) a combination of all the G_{M1} labeled localization patterns (e.g., a ratio of sperm displaying a capacitated state to a total number of assigned sperm) is described.

In certain embodiments of the invention described herein, the classifier in the one or more pre-trained fertility classifiers is a logistic regression model (e.g., of the form:

$$f ( X ) = \frac{1}{1 + exp ( - ( {β}_{0} + {\sum }_{j = 1}^{i} {β}_{j} {X}_{j} ) )}$$

wherein:
ƒ(X) is a measure of fertility,
i is a positive integer,
α is parameter determined during training of the pre-trained classifier, and β₀, β₁, . . . , βᵢ are parameters determined during training of the pre-trained classifier, and
each Xⱼ in {X₁, . . . , Xᵢ} is a datum in the data obtained from the sperm sample).

In certain embodiments, a system for training a fertility classifier for characterizing a fertility status of a male is described. In an embodiment, the system comprises at least one processor and memory addressable by the at least one processor, the memory storing at least one program for execution by the at least one processor, the at least one program comprising instructions for:

A) obtaining a training set that comprises data from sperm samples from a plurality of males associated with a known outcome of an attempt at assisted reproduction (e.g., intra-uterine insemination (IUI)), wherein the data from each respective semen sample comprises a ratio between (i) a combination of the AA G_{M1} localization pattern and APM G_{M1} localization pattern and (ii) the combination of all the G_{M1} labeled localization patterns of sperm in the respective semen sample (e.g., a ratio of sperm displaying a capacitated state to a total number of assigned sperm); and

B) training one or more fertility classifiers based on at least a correspondence between the outcome of the assisted reproduction attempt and the corresponding ratio between (i) a combination of the AA G_{M1} localization pattern and APM G_{M1} localization pattern and (ii) the combination of all the G_{M1} labeled localization patterns of sperm in each respective semen sample.

In certain embodiments, a method for identifying a reproductive approach is described. In one embodiment, the method includes determining a Cap-Score in accordance with the present invention, determining a likelihood of pregnancy within three months of natural conception of within three tries of intrauterine insemination using a logistical regression model as described in the present invention, and determining a reproductive approach to achieving pregnancy based on said value.

### BRIEF DESCRIPTION OF THE DRAWINGS

The foregoing summary, as well as the following detailed description of embodiments of the methods and kits for determining male fertility status, will be better understood when read in conjunction with the appended drawings. It should be understood, however, that the invention is not limited to the precise arrangements and instrumentalities shown.

In the drawings:

FIG. 1 shows INTER, APM, AA, PAPM, AA/PA, ES, and DIFF localization patterns of G_{M1} in normal human sperm and sperm from infertile males under non-capacitating conditions or capacitating conditions;

FIG. 2A shows the relative distributions of the INTER, APM, AA, PAPM, AA/PA, ES, and DIFF localization patterns of G_{M1} in normal human sperm under non-capacitating conditions;

FIG. 2B shows the relative distributions of the INTER, APM, AA, PAPM, AA/PA, ES, and DIFF localization patterns of G_{M1} in normal human sperm under capacitating conditions;

FIG. 2C shows the relative distributions of the INTER, APM, AA, PAPM, AA/PA, ES, and DIFF localization patterns of G_{M1} in human sperm from infertile males under capacitating conditions;

FIG. 3 shows the relative number of the combined APM and AA localizations patterns as a function of time of incubation in human sperm for a group normal males and in human sperm for a group infertile males, under capacitating conditions and non-capacitating conditions, and the clinical outcomes for each group of males;

FIG. 4 shows the percentage of AA and APM localization patterns in sperm from known fertile donors incubated with stimuli promoting capacitation;

FIG. 5 shows a comparison of the percentage of AA and APM localization patterns in sperm from suspected sub-fertile/infertile donors with the statistical thresholds of fertile men; and

FIGS. 6A, 6B, 6C, and 6D show Lined-Cell G_{M1} localization patterns of G_{M1} in sperm from infertile males under capacitating conditions.

FIG. 7 illustrates a logistic regression model of male fertility based on the multi-clinic assisted reproduction outcome training set described in Example 7.

FIGS. 8A, 8B, 8C, and 8D show the use of logistic regression to demonstrate the strong relationship between Cap-Score™ and the probability of generating a pregnancy within three attempts of intrauterine insemination (PGP). FIG. 8A shows data from Example 7, PGP=1/[1+exp[−[−2.863+0.0776*Cap-Score]]]; n=124; p<0.01. To test the fit of the model, an additional 128 data points were added, for a total of n=252. FIG. 8B shows the data from Example 8, PGP=1/[1+exp[−[−2.263+0.0593*Cap-Score]]]; p<0.001. Only a small average change was observed (average 2.6%) for any Cap-Score™, and the fit of the model improved. FIGS. 8C and 8D illustrate the results of PGP versus observed pregnancies within 3 attempts of intrauterine insemination.

### DETAILED DESCRIPTION OF THE DISCLOSURE

With reference to the accompanying drawings, various embodiments of the present invention are described more fully below. Some but not all embodiments of the present invention are shown. Indeed, various embodiments of the invention may be embodied in many different forms and should not be construed as limited to the embodiments expressly described. It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Each and every reference identified herein is incorporated by reference in its entirety.

Unless specifically set forth herein, the terms “a”, “an” and “the” are not limited to one element but instead should be read as meaning “at least one”.

“About” is understood to mean the range of + and 10% of the value referenced. However, use of “about” in reference to a value does not exclude the possibility of the referenced value alone. For example, “about 1 hour” is understood to fully support “54 minutes,” “1 hour,” and “66 minutes.”

The present disclosure is based on the observations that certain G_{M1} localization patterns can provide information regarding male fertility status. Determination of G_{M1} localization patterns is described in U.S. Pat. Nos. 7,160,676, 7,670,763, and 8,367,313, the disclosures of which are incorporated herein by reference. This disclosure provides methods and kits for determination of male fertility status. In certain embodiments, the method is based on a change in the percentage of certain G_{M1} localization patterns upon exposure to in vitro capacitating stimuli. In other embodiments, the method is based specifically on a change in the percentage of a Lined-Cell G_{M1} localization pattern upon exposure to in vitro capacitating stimuli.

In one embodiment, disclosed herein is a method for determining male fertility status. In one embodiment, the method includes subjecting a sperm sample from an individual to in vitro capacitating and in vitro non-capacitating conditions, determining a change in the percentage of certain G_{M1} localization patterns upon exposure to in vitro capacitating conditions, and based on the level of change, identifying the fertility status.

The term “in vitro capacitated” sperm refers to sperm which have been incubated under conditions which promote the process of capacitation. In one embodiment, capacitation conditions include the presence in the medium of one or more of bicarbonate ions, calcium ions, and a sterol acceptor, e.g., serum albumin or a cyclodextrin. In one embodiment, in vitro capacitation conditions include the presence of bicarbonate and calcium ions in the medium, and the presence of a sterol acceptor. In one embodiment, a sterol acceptor is a mediator of sterol efflux. Capacitated sperm have acquired the ability to undergo acrosome exocytosis and have acquired a hyperactivated pattern of motility. The term “in vitro non-capacitated” sperm refers to sperm which are not incubated with one or more of the above-listed stimuli for capacitation. In one embodiment, non-capacitation conditions include the absence of capacitation conditions. In another embodiment, non-capacitation conditions include the absence of one or more of the stimuli needed for capacitation. Non-capacitated sperm do not undergo acrosome exocytosis induced by a physiological ligand such as the zona pellucida, solubilized proteins from the zona pellucida, or progesterone. In addition, sperm incubated under non-capacitating conditions also will not demonstrate hyperactivated motility.

In one embodiment, capacitation may be induced in vitro by exposure to external stimuli such as bicarbonate and calcium ions, and mediators of sterol efflux, e.g., 2-hydroxy-propyl-β-cyclodextrin, methyl-β-cyclodextrin, serum albumin, high density lipoprotein, phospholipids vesicles, liposomes, etc. In certain embodiments, an identifiable change in the G_{M1} localization pattern is observed when sperm are exposed to one or more of these stimuli in vitro.

In one embodiment, after collection, semen samples are typically processed in some way, including one or more of the following: liquefaction, washing, and/or enrichment. In some embodiments, liquefaction involves allowing the sample to liquefy at room temperature or at 37° C. (or any temperature there between) for various time periods (typically 15-20 minutes, but ranging from 10-60 minutes). Liquefaction is a process through which the seminal plasma converts from a gel into a more fluid/liquid consistency. Seminal plasma will typically liquefy without any manipulation, but with especially viscous samples, or if there is a desire to hasten the process or make a consistent liquefaction protocol by which all samples are handled, individuals might manipulate the sample to achieve liquefaction. In certain embodiments the semen sample is manipulated to decrease semen viscosity by using a wide orifice pipette made of non-metallic material. In some embodiments the wide orifice pipette has a gauge size of at least 18 gauge, 16 gauge or 14 gauge. In some embodiments, the wide orifice pipette has an orifice size of at least 1 mm, 1.2 mm or 1.4 mm. In certain embodiments, one can also achieve liquefaction by adding various reagents which do not damage sperm membrane. Reagents which should be avoided are those that damage sperm membrane. The sperm can be washed by centrifugation and resuspension and subjected to semen analysis, and/or be subjected to one or more selection processes including: layering on top of, and centrifugation through a density gradient; layering on top of, and centrifugation through a density gradient followed by collection of the sperm-enriched fraction followed by resuspension and washing; layering on top of, and centrifugation through a density gradient followed by collection of the sperm-enriched fraction and overlaying on top of that a less dense medium into which motile sperm will swim up; or overlaying a less dense medium on top of the sample and allowing motile sperm to swim up into it.

After initial processing, the sperm can be counted, and a given number of sperm can then be placed into containers (such as tubes) containing in vitro non-capacitating or in vitro capacitating medium to achieve desired final concentrations. In one embodiment, the final typical concentration of sperm is 1,000,000/ml (final concentration ranges might vary from 250,000/ml-250,000,000/ml).

The base medium for incubating the sperm under in vitro non-capacitating and capacitating in vitro conditions can be a physiological buffered solution such as, but not limited to, human tubal fluid (HTF); modified human tubal fluid (mHTF); Whitten's medium; modified Whitten's medium; KSOM; phosphate-buffered saline; HEPES-buffered saline; Tris-buffered saline; Ham's F-10; Tyrode's medium; modified Tyrode's medium; TES-Tris (TEST)-yolk buffer; or Biggers, Whitten and Whittingham (BWW) medium. The base medium can have one or more defined or complex sources of protein and other factors added to it, including fetal cord serum ultrafiltrate, Plasmanate, egg yolk, skim milk, albumin, lipoproteins, or fatty acid binding proteins, either to promote viability or at concentrations sufficient to help induce capacitation. Typical stimuli for capacitation include one or more of the following: bicarbonate (typically at 20-25 mM, with ranges from 5-50 mM), calcium (typically at 1-2 mM, with ranges from 0.1-10 mM), and/or cyclodextrin (typically at 1-3 mM, with ranges from 0.1-20 mM). Cyclodextrins may comprise 2-hydroxy-propyl-β-cyclodextrin and/or methyl-β-cyclodextrin. Incubation temperatures are typically 37° C. (ranging from 30° C.-38° C.), and incubation times are typically 1-4 hours (ranging from 30 minutes to 18 hours), though baseline samples can be taken at the start of the incubation period (“time zero”).

In one embodiment, for generating patterns of G_{M1}, the sperm are washed with a standard base medium (e.g., phosphate-buffered saline, Modified Whitten's medium, or other similar media) and incubated with a labeling molecule for G_{M1} which has a detectable label on it. Since G_{M1} has extracellular sugar residues which can serve as an epitope, it can be visualized without having to fix and permeabilize the cells. However, fixation of the cells results in better preservation of the specimen, easier visualization (compared to discerning patterns in swimming sperm) and allows longer visualization time, while contributing to pattern formation. Various fixatives known for histological study of spermatozoa are within the purview of those skilled in the art. Suitable fixatives include paraformaldehyde, glutaraldehyde, Bouin's fixative, and fixatives comprising sodium cacodylate, calcium chloride, picric acid, tannic acid and like. In one embodiment, paraformaldehyde, glutaraldehyde or combinations thereof are used.

Fixation conditions can range from about 0.004% (weight/volume) paraformaldehyde to about 4% (weight/volume) paraformaldehyde, although about 0.01% to about 1% (weight/volume) paraformaldehyde is typically used. In one embodiment, about 0.005% (weight/volume) paraformaldehyde to about 1% (weight/volume) paraformaldehyde can be used. In one embodiment, about 4% paraformaldehyde (weight/volume), about 0.1% glutaraldehyde (weight/volume) and about 5 mM CaCl₂ in phosphate buffered saline can be used.

The period of time a sperm sample is fixed in a fixative may vary. In one embodiment, a sperm sample is fixed in fixative for about 5 hours or less. In one embodiment, a sperm sample is fixed in a fixative for greater than about 5 hours. In another embodiment, a sperm sample is fixed in a fixative for about 0.5 hours, for about 1 hours, for about 1.5 hours, for about 2 hours, for about 2.5 hours, for about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, about 24.5 hours, about 25 hours, about 25.5 hours, about 26 hours, about 26.5 hours, about 27 hours, about 27.5 hours, about 28 hours, about 28.5 hours, about 29 hours, about 29.5 hours, about 30, or any range determinable from the preceding times (for example, about 26 hours to about 28 hours, or about 3 hours to about 5 hours).

The localization pattern of G_{M1} in live or fixed sperm can be obtained by using labeling binding techniques. A molecule having specific affinity for the G_{M1} ganglioside can be used. The labeling molecule can be directly linked to a detectable label (such as a fluorophore) or may be detected by a second labeling molecule which has a detectable label on it. For example, the b subunit of cholera toxin is known to specifically bind to G_{M1}. Therefore, a labeled (such as fluorescent labeled) cholera toxin b subunit can be used to obtain a pattern of distribution of G_{M1}. In one embodiment, final concentrations of the b subunit of cholera toxin linked to fluorophore are about 10 μg/ml to about 15 μg/ml. In another embodiment, the final concentrations of the b subunit of cholera toxin linked to fluorophore are about 0.1 μg/ml to about 50 μg/ml. Alternatively, a labeled antibody to G_{M1} can be used. In yet another alternative, a labeled antibody to the cholera toxin b subunit can be used to visualize the pattern of G_{M1} staining. And in yet another alternative, a labeled secondary antibody which binds to either the primary antibody that binds directly to G_{M1} or to the primary antibody that binds to the b subunit of cholera toxin could be used. The term “G_{M1} staining” or “staining of G_{M1}” or “labeling” or related terms as used herein means the staining seen on or in cells due to the binding of labeled affinity molecules to G_{M1}. For example, when fluorescent tagged/labeled cholera toxin b subunit is used for localization of GM₁, the signal or staining is from the cholera toxin b subunit but is indicative of the location of G_{M1}. The terms “signal” and “staining” and “labeling” are used interchangeably. The detectable label is such that it is capable of producing a detectable signal. Such labels include a radionuclide, an enzyme, a fluorescent agent or a chromophore. Labeling (or staining) and visualization of G_{M1} distribution in sperm is carried out by standard techniques. Labeling molecules other than the b subunit of cholera toxin can also be used. These include polyclonal and monoclonal antibodies. Specific antibodies to G_{M1} ganglioside can be generated by routine immunization protocols, or can be purchased commercially (e.g., Matreya, Inc., State College, Pa.). The antibodies may be raised against G_{M1} or, can be generated by using peptide mimics of relevant epitopes of the G_{M1} molecule. Identification and generation of peptide mimics is well known to those skilled in the art. In addition, the binding of the b subunit to cholera toxin might be mimicked by a small molecule. Identification of small molecules that have similar binding properties to a given reagent is well known to those skilled in the art.

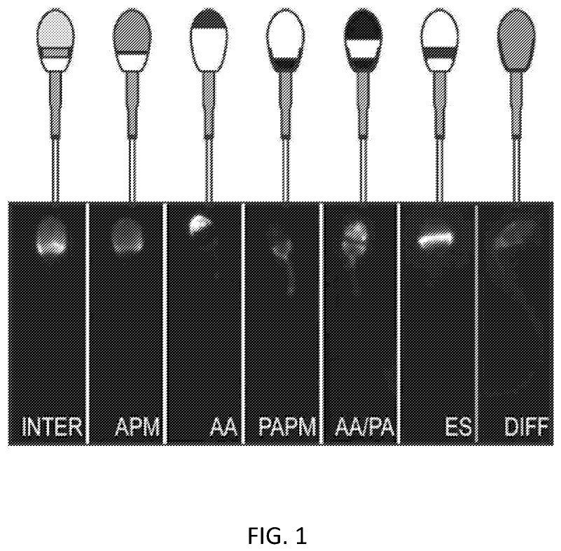

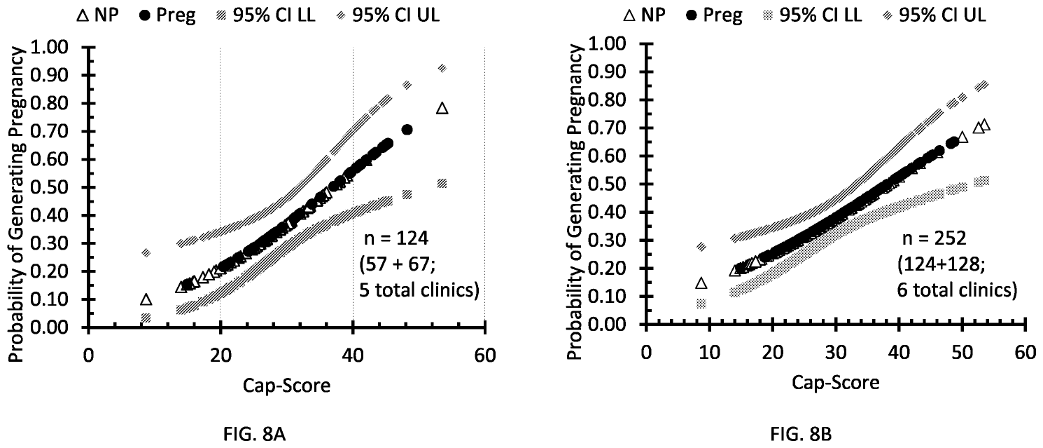

For human sperm, eight different localization patterns (see details under Example 1) were observed when the sperm was under in vitro capacitating conditions. These patterns are designated as INTER, APM, AA, PAPM, AA/PA, ES, DIFF, and Lined Cell. The INTER, APM, AA, PAPM, AA/PA, ES, and DIFF patterns are shown in FIG. 1 and the Lined-Cell pattern is shown in FIGS. 6A, 6B, 6C, and 6D, each of which are further described below:
INTER: The vast majority of the fluorescence is in a band around the equatorial segment, with some signal in the plasma membrane overlying the acrosome. There is usually a gradient of signal, with the most at the equatorial segment and then progressively less toward the tip. There is often an increase in signal intensity on the edges of the sperm head in the band across the equatorial segment.
APM (Acrosomal Plasma Membrane): Compared to INTER there is less distinction in this pattern between the equatorial signal and that moving toward the apical tip. That is, the signal in the plasma membrane overlying the acrosome is more evenly distributed. APM signal is seen either from the bright equatorial INTER band moving apically toward the tip, or it can start further up toward the tip and be found in a smaller region, as it is a continuum with the AA.
AA (Apical Acrosome): In this pattern, the fluorescence is becoming more and more concentrated toward the apical tip, increased in brightness and reduced in area with signal.
PAPM (Post Acrosomal Plasma Membrane): Signal is exclusively in the post-acrosomal plasma membrane.
AA/PA (Apical Acrosome/Post Acrosome): Signal is both in the plasma membrane overlying the acrosome and post-acrosomal plasma membrane. Signal is missing from the equatorial segment.
ES (Equatorial Segment): Bright signal is seen solely in the equatorial segment. It may be accompanied by thickening of the sperm head across the equatorial region.
DIFF (Diffuse): Diffuse signal is seen over the whole sperm head.
Lined-Cell: Signal is seen at the top of the post-acrosomal region and at the plasma membrane overlying the acrosome as well as the bottom of the equatorial segment (i.e., the post acrosome/equatorial band). Signal is missing around the equatorial segment.

The term “G_{M1} localization pattern” is used interchangeably with “pattern” or “localization pattern.”

FIGS. 6A, 6B, 6C, and 6D show Lined-Cell G_{M1} localization patterns of G_{M1} in sperm from infertile males under capacitating conditions. Specifically, FIG. 6A shows a Lined-Cell G_{M1} localization pattern where the signal is evenly distributed at the post acrosome/equatorial band and at the plasma membrane overlying the acrosome. FIG. 6B shows a Lined-Cell G_{M1} localization pattern where the signal at the plasma membrane overlying the acrosome is brighter than the signal at the post acrosome/equatorial band. FIGS. 6C and 6D show a signal at the post acrosome/equatorial band that is brighter than the signal at the plasma membrane overlying the acrosome.

It was observed that while the INTER, AA, APM patterns, and combinations of these patterns, correlate positively with viable sperm with normal sperm membrane architecture and therefore fertility, the PAPM, AA/PA, ES, DIFF, and the Lined-Cell patterns do not positively correlate with viability, normal membrane architecture and fertility. If incubated under non-capacitating conditions, the majority of viable sperm with normal membrane architecture will exhibit the INTER pattern, which is characterized by the majority of labeling being near the equatorial segment, with the rest extending through the plasma membrane overlying the acrosome. Additionally, there is an increase in the number of the APM and AA patterns upon exposure to stimuli for capacitation. The APM pattern shows more uniform signal in the plasma membrane overlying the acrosome, whereas the AA pattern shows increasing intensity of signal in the rostral part of the sperm head, the apical acrosome, and reduced signal moving caudally toward the equatorial segment. Sperm incubated under in vitro non-capacitated conditions for infertile individuals have G_{M1} localization patterns that are similar to G_{M1} localization patterns of sperm incubated under in vitro non-capacitated conditions for normal individuals.

In one embodiment disclosed herein is a method for determining male fertility status. In one embodiment, the method includes the steps of exposing a first portion of a sperm sample from a male to non-capacitating conditions to obtain an in vitro non-capacitated sperm sample; exposing a second portion of the sperm sample to capacitating conditions to obtain an in vitro capacitated sperm sample; fixing the in vitro non-capacitated sperm sample and the in vitro capacitated sperm sample with a fixative for a time period of at least: one hour, two hours, three hours, four hours, five hours, six hours, seven hours, eight hours, nine hours, ten hours, eleven hours, twelve hours, eighteen hours or twenty four hours, treating the fixed in vitro non-capacitated sperm sample and the fixed in vitro capacitated sperm sample with a labeling molecule for G_{M1} localization patterns, wherein the labeling molecule has a detectable label; identifying more than one labeled G_{M1} localization patterns for the labeled fixed in vitro non-capacitated sperm sample and the labeled fixed in vitro capacitated sperm sample, said G_{M1} labeled localization patterns being an apical acrosome (AA) G_{M1} localization pattern, an acrosomal plasma membrane (APM) G_{M1} localization pattern, a Lined-Cell G_{M1} localization pattern and all other labeled G_{M1} localization patterns; comparing the labeled G_{M1} localization patterns for the labeled fixed in vitro non-capacitated sperm sample to the labeled G_{M1} localization patterns for the labeled fixed in vitro capacitated sperm sample; based on the comparison, assigning the apical acrosome (AA) G_{M1} localization pattern and the acrosomal plasma membrane (APM) G_{M1} localization pattern to a capacitated state and assigning the Lined-Cell G_{M1} localization pattern and all other labeled G_{M1} localization patterns to a non-capacitated state; and characterizing a fertility status of the male based on the identified G_{M1} labeled localization patterns for the labeled fixed in vitro non-capacitated sperm sample and the labeled fixed in vitro capacitated sperm sample. In one embodiment, the characterizing step comprises the steps of: determining a fertility threshold associated with a percentage of [(AA G_{M1} localization patterns plus APM G_{M1} localization patterns)/total G_{M1} localization patterns] for the labeled fixed in vitro capacitated sperm sample; wherein a reference percentage of [(AA G_{M1} localization patterns plus APM G_{M1} localization patterns)/total G_{M1} localization patterns], based on distribution statistics of a known fertile population corresponding to: greater than a percentage that is one standard deviation below the reference mean percentage indicates fertile; less than a percentage that is one standard deviation below the reference mean percentage and greater than a percentage that is two standard deviations below the reference mean percentage indicates sub-fertile; less than a percentage that is two standard deviations below the reference mean percentage indicates infertile; comparing the percentage of [(AA G_{M1} localization patterns plus APM G_{M1} localization patterns)/total G_{M1} localization patterns] for the labeled fixed in vitro capacitated sperm sample to the reference percentage of [(AA G_{M1} localization patterns plus APM G_{M1} localization patterns)/total G_{M1} localization patterns] and identifying the fertility threshold based on the comparison.

In another embodiment, a fertility threshold associated with a percentage of [(AA G_{M1} localization patterns plus APM G_{M1} localization patterns)/total G_{M1} localization patterns] is determined, wherein a reference percentage of [(AA G_{M1} localization patterns plus APM G_{M1} localization patterns)/total G_{M1} localization patterns], based on distribution statistics of a known fertile population corresponding to: greater than a percentage that is one standard deviation below the reference mean percentage indicates normal male fertility; less than a percentage that is one standard deviation below the reference mean percentage indicates abnormal male fertility. The percentage of [(AA G_{M1} localization patterns plus APM G_{M1} localization patterns)/total G_{M1} localization patterns] for the labeled fixed in vitro capacitated sperm sample is compared to the reference percentage of [(AA G_{M1} localization patterns plus APM G_{M1} localization patterns)/total G_{M1} localization patterns]. The fertility threshold is identified based on the comparison.

In one embodiment, prior to the exposing steps, a semen sample is treated to decrease semen viscosity using a wide orifice pipette made of non-metallic material and using a reagent that does not damage sperm membrane chosen from the various reagents that are used to decrease semen viscosity. In some embodiments, the membrane damaging reagent is selected from the group consisting of (i) a protease; (ii) a nuclease (iii) a mucolytic agent; (iv) a lipase; (v) an esterase and (vi) glycoside hydrolases. In some embodiments, the identifying step is repeated until the number of Lined-Cell G_{M1} localization patterns is substantially constant. In one such embodiment, after the identifying step is performed, determining the number of Lined-Cell G_{M1} localization patterns, for the labeled fixed in vitro capacitated sperm until the number is less than 5%, less than 3% of the total number of labeled cells; or ranges from 1% to 5%, 2 to 5% of the total number of labeled cells. In another such embodiment, after the identifying step is performed, determining the number of Lined-Cell G_{M1} localization patterns, for the labeled fixed in vitro non-capacitated sperm until the number is less than: 25%, 20%, 15% or 10% of the total number of labeled cells; or ranges from 2% to 25%; 2% to 20%; 2 to 15%; 2 to 10%; 2 to 5% of the total number of labeled cells. In some embodiments the wide orifice pipette has a gauge size of at least 18 gauge, 16 gauge or 14 gauge. In some embodiments, the wide orifice pipette has an orifice size of at least 1 mm, 1.2 mm or 1.4 mm.

In one such embodiment, the characterizing step may include the steps of: determining the number of each G_{M1} labeled localization patterns for a predetermined number of the labeled fixed in vitro non-capacitated sperm sample; determining the number of each G_{M1} labeled localization patterns for a predetermined number of the labeled fixed in vitro capacitated sperm sample; calculating a ratio for a sum of the number of AA G_{M1} localization patterns and number of APM G_{M1} localization patterns over a sum of the total number of G_{M1} labeled localization patterns for the labeled fixed in vitro non-capacitated sperm sample; and calculating a ratio for a sum of the number of AA G_{M1} localization patterns and number of APM G_{M1} localization patterns over a sum of the total number of G_{M1} labeled localization patterns for the labeled fixed in vitro capacitated sperm sample.

In one such embodiment, the method may further include the steps of: comparing the ratio for the labeled fixed in vitro non-capacitated sperm to a ratio of labeled fixed in vitro non-capacitated sperm having a known fertility status; and comparing the ratio for the labeled fixed in vitro capacitated sperm to a ratio of labeled fixed in vitro capacitated sperm having a known fertility status.

In one embodiment disclosed herein is a method for determining male fertility status. In one embodiment, the method includes the steps of: obtaining a first portion of a sperm sample from a male that has been exposed to in vitro non-capacitating conditions, fixed in a fixative for at least: one hour, two hours, three hours, four hours, five hours, six hours, seven hours, eight hours, nine hours, ten hours, eleven hours, twelve hours, eighteen hours or twenty four hours, and treated with a labeling molecule for G_{M1} localization patterns, wherein the labeling molecule has a detectable label; obtaining a second portion of the sperm sample that has been exposed to in vitro capacitating conditions, fixed in a fixative, and treated with the labeling molecule for G_{M1} localization patterns; identifying more than one G_{M1} labeled localization patterns for the labeled fixed in vitro non-capacitated sperm sample and the labeled fixed in vitro capacitated sperm sample, said G_{M1} labeled localization patterns being an apical acrosome (AA) G_{M1} localization pattern, an acrosomal plasma membrane (APM) G_{M1} localization pattern, a Lined-Cell G_{M1} localization pattern and all other labeled G_{M1} localization patterns; comparing the labeled G_{M1} localization patterns for the labeled fixed in vitro non-capacitated sperm sample to the labeled G_{M1} localization patterns for the labeled fixed in vitro capacitated sperm sample; based on the comparison, assigning the apical acrosome (AA) G_{M1} localization pattern and the acrosomal plasma membrane (APM) G_{M1} localization pattern to a capacitated state and assigning the Lined-Cell G_{M1} localization pattern and all other labeled G_{M1} localization patterns to a non-capacitated state; and characterizing a fertility status of the male based on the identified G_{M1} labeled localization patterns for the labeled fixed in vitro non-capacitated sperm sample and the labeled fixed in vitro capacitated sperm sample. In one embodiment, the characterizing step comprises the steps of: determining a fertility threshold associated with a percentage of [(AA G_{M1} localization patterns plus APM G_{M1} localization patterns)/total G_{M1} localization patterns] for the labeled fixed in vitro capacitated sperm sample; wherein a reference percentage of [(AA G_{M1} localization patterns plus APM G_{M1} localization patterns)/total G_{M1} localization patterns], based on distribution statistics of a known fertile population corresponding to: greater than a percentage that is standard deviation below the reference mean percentage indicates fertile; less than a percentage that is one standard deviation below the reference mean percentage and greater than a percentage that is two standard deviations below the reference mean percentage indicates sub-fertile; less than a percentage that is two standard deviations below the reference mean percentage indicates infertile; comparing the percentage of [(AA G_{M1} localization patterns plus APM G_{M1} localization patterns)/total G_{M1} localization patterns] for the labeled fixed in vitro capacitated sperm sample to the reference percentage of [(AA G_{M1} localization patterns plus APM G_{M1} localization patterns)/total G_{M1} localization patterns] and identifying the fertility threshold based on the comparison.

In another embodiment, a fertility threshold associated with a percentage of [(AA G_{M1} localization patterns plus APM G_{M1} localization patterns)/total G_{M1} localization patterns] is determined, wherein a reference percentage of [(AA G_{M1} localization patterns plus APM G_{M1} localization patterns)/total G_{M1} localization patterns], based on distribution statistics of a known fertile population corresponding to: greater than a percentage that is one standard deviation below the reference mean percentage indicates normal male fertility; less than a percentage that is one standard deviation below the reference mean percentage indicates abnormal male fertility. The percentage of [(AA G_{M1} localization patterns plus APM G_{M1} localization patterns)/total G_{M1} localization patterns] for the labeled fixed in vitro capacitated sperm sample is compared to the reference percentage of [(AA G_{M1} localization patterns plus APM G_{M1} localization patterns)/total G_{M1} localization patterns]. The fertility threshold is identified based on the comparison.

In some embodiments, the identifying step is repeated until the number of Lined-Cell G_{M1} localization patterns is substantially constant. In one such embodiment, after the identifying step is performed, determining the number of Lined-Cell G_{M1} localization patterns, for the labeled fixed in vitro capacitated sperm until the number is less than 5%, less than 3% of the total number of labeled cells; or ranges from 1% to 5%, 2 to 5% of the total number of labeled cells. In another such embodiment, after the identifying step is performed, determining the number of Lined-Cell G_{M1} localization patterns, for the labeled fixed in vitro non-capacitated sperm until the number is less than: 25%, 20%, 15% or 10% of the total number of labeled cells; or ranges from 2% to 25%; 2% to 20%; 2 to 15%; 2 to 10%; 2 to 5% of the total number of labeled cells.

In one embodiment of such method, the method further includes the steps of: determining the number of each G_{M1} labeled localization patterns for a predetermined number of the labeled fixed in vitro non-capacitated sperm sample and the labeled fixed in vitro capacitated sperm sample, and calculating a ratio for a sum of the number of AA G_{M1} localization patterns and number of APM G_{M1} localization patterns over a sum of the total number of G_{M1} localization patterns for each of the labeled fixed in vitro non-capacitated sperm sample and the labeled fixed in vitro capacitated sperm sample.

In one such embodiment, the characterizing step may further include the steps of: comparing the ratio for the labeled fixed in vitro capacitated sperm sample to ratios of G_{M1} localization patterns of in vitro capacitated sperm for males having a known fertility status; and comparing the ratio for the labeled fixed in vitro non-capacitated sperm sample to ratios of G_{M1} localization patterns in vitro non-capacitated sperm for males having a known fertility status.

In one embodiment disclosed herein is a method for determining male fertility status. In one embodiment, the method includes the steps of: exposing, in vitro, a sperm sample from a male to capacitating conditions; fixing the capacitated sperm sample with a fixative for at least: one hour, two hours, three hours, four hours, five hours, six hours, seven hours, eight hours, nine hours, ten hours, eleven hours, twelve hours, eighteen hours or twenty four hours, treating the fixed in vitro capacitated sperm sample with a labeling molecule for G_{M1} localization patterns, wherein the labeling molecule has a detectable label; identifying more than one G_{M1} labeled localization patterns for the labeled fixed in vitro capacitated sperm sample, said G_{M1} labeled localization patterns being an apical acrosome (AA) G_{M1} localization pattern, an acrosomal plasma membrane (APM) G_{M1} localization pattern, a Lined-Cell G_{M1} localization pattern and all other labeled G_{M1} localization patterns; assigning the apical acrosome (AA) G_{M1} localization pattern and the acrosomal plasma membrane (APM) G_{M1} localization pattern to a capacitated state and assigning the Lined-Cell G_{M1} localization pattern and all other labeled G_{M1} localization patterns to a non-capacitated state; and characterizing a fertility status of the male. In one embodiment, the characterizing step comprises the steps of: determining a fertility threshold associated with a percentage of [(AA G_{M1} localization patterns plus APM G_{M1} localization patterns)/total G_{M1} localization patterns] for the labeled fixed in vitro capacitated sperm sample; wherein a reference percentage of [(AA G_{M1} localization patterns plus APM G_{M1} localization patterns)/total G_{M1} localization patterns], based on distribution statistics of a known fertile population corresponding to: greater than a percentage that is one standard deviation below the reference mean percentage indicates fertile; less than a percentage that is one standard deviation below the reference mean percentage and greater than a percentage that is two standard deviations below the reference mean percentage indicates sub-fertile; less than a percentage that is two standard deviations below the reference mean percentage indicates infertile; comparing the percentage of [(AA G_{M1} localization patterns plus APM G_{M1} localization patterns)/total G_{M1} localization patterns] for the labeled fixed in vitro capacitated sperm sample to the reference percentage of [(AA G_{M1} localization patterns plus APM G_{M1} localization patterns)/total G_{M1} localization patterns] and identifying the fertility threshold based on the comparison.

In another embodiment, a fertility threshold associated with a percentage of [(AA G_{M1} localization patterns plus APM G_{M1} localization patterns)/total G_{M1} localization patterns] is determined, wherein a reference percentage of [(AA G_{M1} localization patterns plus APM G_{M1} localization patterns)/total G_{M1} localization patterns], based on distribution statistics of a known fertile population corresponding to: greater than a percentage that is one standard deviation below the reference mean percentage indicates normal male fertility; less than one standard deviation below the reference mean percentage indicates abnormal male fertility. The percentage of [(AA G_{M1} localization patterns plus APM G_{M1} localization patterns)/total G_{M1} localization patterns] for the labeled fixed in vitro capacitated sperm sample is compared to the reference percentage of [(AA G_{M1} localization patterns plus APM G_{M1} localization patterns)/total G_{M1} localization patterns]. The fertility threshold is identified based on the comparison.

In one embodiment, prior to the exposing steps, a semen sample is treated to decrease semen viscosity using a wide orifice pipette made of non-metallic material and using a reagent that does not damage sperm membrane chosen from the various reagents that are used to decrease semen viscosity. In some embodiments, the membrane damaging reagent is selected from the group consisting of (i) a protease; (ii) a nuclease (iii) a mucolytic agent; (iv) a lipase; (v) an esterase and (vi) glycoside hydrolases. In some embodiments, the identifying step is repeated until the number of Lined-Cell G_{M1} localization patterns is substantially constant. In one such embodiment, after the identifying step is performed, determining the number of Lined-Cell G_{M1} localization patterns, for the labeled fixed in vitro capacitated sperm until the number is less than 5%, less than 3% of the total number of labeled cells; or ranges from 1% to 5%, 2 to 5% of the total number of labeled cells. In another such embodiment, after the identifying step is performed, determining the number of Lined-Cell G_{M1} localization patterns, for the labeled fixed in vitro non-capacitated sperm until the number is less than: 25%, 20%, 15% or 10% of the total number of labeled cells; or ranges from 2% to 25%; 2% to 20%; 2 to 15%; 2 to 10%; 2 to 5% of the total number of labeled cells. In some embodiments the wide orifice pipette has a gauge size of at least 18 gauge, 16 gauge or 14 gauge. In some embodiments, the wide orifice pipette has an orifice size of at least 1 mm, 1.2 mm or 1.4 mm.

In one embodiment of such method, the method may further include the steps of: comparing the ratio of G_{M1} localization patterns to ratios of G_{M1} localization patterns for males having a known fertility status. In one embodiment, the known fertility status corresponds to fertile males. In another embodiment, the known fertility status corresponds to infertile males. In one such embodiment, the comparing step includes the steps of: determining the number of each G_{M1} labeled localization patterns for a predetermined number of the labeled fixed in vitro capacitated sperm sample, and calculating a ratio for a sum of the number of AA G_{M1} localization patterns and number of APM G_{M1} localization patterns over a sum of the total number of G_{M1} labeled localization patterns.

In one embodiment disclosed herein is a method for determining male fertility status. In one embodiment, the method includes the steps of: obtaining a first portion of a sperm sample from a male that has been exposed to in vitro capacitating conditions, fixed in a fixative for at least: one hour, two hours, three hours, four hours, five hours, six hours, seven hours, eight hours, nine hours, ten hours, eleven hours, twelve hours, eighteen hours or twenty four hours, and stained with a labeling molecule for G_{M1} localization patterns, wherein the labeling molecule has a detectable label; identifying more than one G_{M1} labeled localization patterns for the labeled fixed in vitro capacitated sperm sample, said G_{M1} localization patterns being an apical acrosome (AA) G_{M1} localization pattern, an acrosomal plasma membrane (APM) G_{M1} localization pattern, a Lined-Cell G_{M1} localization pattern and all other labeled G_{M1} localization patterns; assigning the apical acrosome (AA) G_{M1} localization pattern and the acrosomal plasma membrane (APM) G_{M1} localization pattern to a capacitated state and assigning the Lined-Cell G_{M1} localization pattern and all other labeled G_{M1} localization patterns to a non-capacitated state; and characterizing a fertility status of the male. In one embodiment, the characterizing step comprises the steps of: determining a fertility threshold associated with a percentage of [(AA G_{M1} localization patterns plus APM G_{M1} localization patterns)/total G_{M1} localization patterns] for the labeled fixed in vitro capacitated sperm sample; wherein a reference percentage of [(AA G_{M1} localization patterns plus APM G_{M1} localization patterns)/total G_{M1} localization patterns], based on distribution statistics of a known fertile population corresponding to: greater than a percentage that is one standard deviation below the reference mean percentage indicates fertile; less than a percentage that is one standard deviation below the reference mean percentage and greater than a percentage that is that is two standard deviations below the reference mean percentage indicates sub-fertile; less than a percentage that is two standard deviations below the reference mean percentage indicates infertile; comparing the percentage of [(AA G_{M1} localization patterns plus APM G_{M1} localization patterns)/total G_{M1} localization patterns] for the labeled fixed in vitro capacitated sperm sample to the reference percentage of [(AA G_{M1} localization patterns plus APM G_{M1} localization patterns)/total G_{M1} localization patterns] and identifying the fertility threshold based on the comparison.

In another embodiment, a fertility threshold associated with a percentage of [(AA G_{M1} localization patterns plus APM G_{M1} localization patterns)/total G_{M1} localization patterns] is determined, wherein a reference percentage of [(AA G_{M1} localization patterns plus APM G_{M1} localization patterns)/total G_{M1} localization patterns], based on distribution statistics of a known fertile population corresponding to: greater than a percentage that is one standard deviation below the reference mean percentage indicates normal male fertility; less than a percentage that is one standard deviation below the reference mean percentage indicates abnormal male fertility. The percentage of [(AA G_{M1} localization patterns plus APM G_{M1} localization patterns)/total G_{M1} localization patterns] for the labeled fixed in vitro capacitated sperm sample is compared to the reference percentage of [(AA G_{M1} localization patterns plus APM G_{M1} localization patterns)/total G_{M1} localization patterns]. The fertility threshold is identified based on the comparison.

In some embodiments, the identifying step is repeated until the number of Lined-Cell G_{M1} localization patterns is substantially constant. In one such embodiment, after the identifying step is performed, determining the number of Lined-Cell G_{M1} localization patterns, for the labeled fixed in vitro capacitated sperm until the number is less than 5%, less than 3% of the total number of labeled cells; or ranges from 1% to 5%, 2 to 5% of the total number of labeled cells. In another such embodiment, after the identifying step is performed, determining the number of Lined-Cell G_{M1} localization patterns, for the labeled fixed in vitro non-capacitated sperm until the number is less than: 25%, 20%, 15% or 10% of the total number of labeled cells; or ranges from 2% to 25%; 2% to 20%; 2 to 15%; 2 to 10%; 2% to 5% of the total number of labeled cells.

In one embodiment of such method, the method may further include the steps of: comparing the ratio of G_{M1} localization patterns to ratios of G_{M1} localization patterns for males having a known fertility status. In one embodiment, the known fertility status corresponds to fertile males. In another embodiment, the known fertility status corresponds to infertile males. In one such embodiment, the comparing step includes the steps of: determining the number of each G_{M1} labeled localization patterns for a predetermined number of the labeled fixed in vitro capacitated sperm sample, and calculating a ratio for a sum of the number of AA G_{M1} localization patterns and number of APM G_{M1} localization patterns over a sum of the total number of G_{M1} labeled localization patterns.

In one embodiment disclosed herein is a method for determining male fertility status. In one embodiment, the method includes the steps of: obtaining a sperm sample, wherein at least a portion of the sperm sample has been exposed to in vitro capacitating conditions to obtain in vitro capacitated sperm, has been exposed to a fixative for at least: one hour, two hours, three hours, four hours, five hours, six hours, seven hours, eight hours, nine hours, ten hours, eleven hours, twelve hours, eighteen hours or twenty four hours, and has been stained for G_{M1}; obtaining values for one or more semen parameters of the sperm sample; determining a Cap-Score of the labeled fixed in vitro capacitated sperm sample based on one or more G_{M1} labeled localization patterns, said G_{M1} labeled localization patterns being an apical acrosome (AA) G_{M1} localization pattern, a post-acrosomal plasma membrane (APM) G_{M1} localization pattern, a Lined-Cell G_{M1} localization pattern and all other labeled G_{M1} localization patterns; and calculating a male fertility index (WI) value of the male based on the determined CAP score and the one or more obtained semen parameters. In one embodiment, the one or more semen parameters of the sperm sample are selected from the group consisting of volume of the original sperm sample, concentration of sperm, motility of sperm, and morphology of sperm.

An embodiment disclosed herein is a method for determining male fertility status. In one embodiment, the method comprises the following steps. A sample of in vitro capacitated sperm cells is treated with a fluorescence label. One or more capacitated-fluorescence images is obtained wherein the images display one or more G_{M1} localization patterns associated with fluorescence labeled in vitro capacitated sperm cells. An apical acrosome (AA) G_{M1} localization pattern and an acrosomal plasma membrane (APM) G_{M1} localization pattern are each assigned to a capacitated state and a Lined-Cell G_{M1} localization pattern and all other labeled G_{M1} localization patterns are assigned to a non-capacitated state each displayed in the cap-fluorescence images. A number for G_{M1} localization patterns is measured, the patterns comprising AA G_{M1} localization pattern, APM G_{M1} localization pattern, Lined-Cell G_{M1} localization pattern and all other labeled G_{M1} localization patterns, for the fluorescence labeled in vitro capacitated sperm cells, displayed in the capacitated-fluorescence images to determine a percentage of [(AA G_{M1} localization patterns plus APM G_{M1} localization patterns)/total G_{M1} localization patterns]. A fertility threshold associated with a percentage of [(AA G_{M1} localization patterns plus APM G_{M1} localization patterns)/total G_{M1} localization patterns] is determined, wherein a reference percentage of [(AA G_{M1} localization patterns plus APM G_{M1} localization patterns)/total G_{M1} localization patterns] corresponding to: greater than a percentage that is one standard deviation below a reference mean percentage indicates fertile; less than a percentage that is one standard deviation below a reference mean percentage and greater than a percentage that is two standard deviations below a reference mean percentage indicates sub-fertile; less than a percentage that is two standard deviations below a reference mean percentage indicates infertile. The percentage of [(AA G_{M1} localization patterns plus APM G_{M1} localization patterns)/total G_{M1} localization patterns] is compared to the reference percentage of [(AA G_{M1} localization patterns plus APM G_{M1} localization patterns)/total G_{M1} localization patterns]. The fertility threshold is identified based on the comparison.

In another embodiment, a fertility threshold associated with a percentage of [(AA G_{M1} localization patterns plus APM G_{M1} localization patterns)/total G_{M1} localization patterns] is determined, wherein a reference percentage of [(AA G_{M1} localization patterns plus APM G_{M1} localization patterns)/total G_{M1} localization patterns], based on distribution statistics of a known fertile population corresponding to: greater than a percentage that is one standard deviation below the reference mean percentage indicates normal male fertility; less than a percentage that is one standard deviation below the reference mean percentage indicates abnormal fertility. The percentage of [(AA G_{M1} localization patterns plus APM G_{M1} localization patterns)/total G_{M1} localization patterns] for the labeled fixed in vitro capacitated sperm sample is compared to the reference percentage of [(AA G_{M1} localization patterns plus APM G_{M1} localization patterns)/total G_{M1} localization patterns]. The fertility threshold is identified based on the comparison.

In one such embodiment, the identifying step is also based on one or more of the following: patient demographics, reproductive status of female partner, sperm concentration, total motility, progressive motility, semen volume, semen pH, semen viscosity and/or sperm morphology and combinations thereof.

In various embodiments of the methods described herein, the sperm cells are treated in vitro with capacitation conditions for a capacitation time period of: at least one hour; at least 2 hours; at least 3 hours; at least 12 hours; at least 18 hours; at least 24 hours; for a capacitation time period ranging between 0.5 hours to 3 hours; 3 hours to 12 hours; 6 hours to 12 hours; 3 hours to 24 hours; 12 hours to 24 hours; or 18 hours to 24 hours.

In various embodiments of the methods described herein, the in vitro capacitated sperm cells are treated with a fixative for a fixative time period of: at least 0.5 hour; at least 3 hours; at least 12 hours; at least 18 hours; at least 24 hours; at least 30 hours; at least 36 hours; or at least 48 hours, for a fixation time period ranging between 0.5 hours to 3 hours; 3 hours to 12 hours; 6 hours to 12 hours; 3 hours to 18 hours; 6-18 hours; 6-24 hours; 12 hours to 24 hours; 18 hours to 24 hours; 18-30 hours; 18-36 hours; 24-30 hours; 24-26 hours; 18-48 hours; 24-48 hours; or 36-48 hours.

In various embodiments of the methods described herein, the more than one of G_{M1} labeled localization patterns comprises AA G_{M1} localization pattern, APM G_{M1} localization pattern, Lined-Cell G_{M1} localization pattern, intermediate (INTER) G_{M1} localization pattern, post acrosomal plasma membrane (PAPM) G_{M1} localization pattern, apical acrosome/post acrosome (AA/PA) G_{M1} localization pattern, equatorial segment (ES) G_{M1} localization pattern, and diffuse (DIFF) G_{M1} localization pattern.

In one embodiment, exposing the first portion of the sperm sample to non-capacitating conditions and exposing the second portion of the sperm sample to capacitating conditions occur concurrently.

The male individual may be a human or a non-human animal. In the case of a non-human animal, identification of patterns that are correlated with fertility status can be carried out based on the teachings provided herein. Non-human animals include horse, cattle, dog, cat, swine, goat, sheep, deer, rabbit, chicken, turkey, various species of fish and various zoological species.

In one embodiment, the method of this disclosure provides a method for designating a male as likely infertile comprising obtaining G_{M1} localization patterns (e.g., one or more of Lined-Cell, AA, APM, and all other G_{M1} localization patterns) in the sperm from the individual and from a normal control that have been incubated under capacitating and non-capacitating conditions and optionally fixed, and comparing the G_{M1} localization patterns. In the normal control, a statistically significant change in the percentage of sperm displaying certain localization patterns would be observed. If the same change is not observed in the sperm from the test individual, then the individual is designated as having an abnormal fertility status. In one embodiment, the patterns that are informative of normal and abnormal fertility status are localization patterns Lined-Cell, INTER, AA and/or APM. Thus, in a sample from an individual who is known to have a normal fertility status (which may be used as a control), there is a higher percentage of sperm exhibiting AA and/or APM localization patterns, and a lower percentage of sperm exhibiting the Lined-Cell and/or INTER localization pattern upon exposure to in vitro capacitating conditions when compared to the sperm being exposed to in vitro non-capacitating conditions. If no difference or no significant difference is observed in the percentages of one or more of these localization patterns upon exposure to in vitro capacitating conditions as compared to when the sperm is exposed to in vitro non-capacitating conditions, then the individual is designated as having fertility problems. In a variation of the above embodiment, the control may be from an individual known to be infertile or sub-fertile. In this embodiment, if the changes in G_{M1} patterns from the test individual upon in vitro capacitation in the Lined-Cell, INTER, AA and/or APM localization patterns are the same as the control, then the individual can be deemed as sub-fertile or infertile.

In yet another variation of the above embodiment, the sample from a test individual may be evaluated without comparing to a control. If no change, or no significant change, is observed in the number of Lined-Cell, INTER, AA and/or APM patterns upon exposure to in vitro capacitating conditions, then the individual may be deemed as abnormal and may be designated for further testing, whereas if changes are observed such that Lined-Cell and/or INTER is decreased, AA is increased, and/or APM is increased, then the individual may be designated as having normal fertility.

In one embodiment, the method comprises analysis of G_{M1} localization patterns to identify number of AA and APM patterns in sperm exposed to in vitro capacitating conditions. The number can be expressed as a percentage of one or more of the G_{M1} distribution patterns relative to the total. In one embodiment, fertility is predicted based on a comparison of the number of AA and/or APM localization patterns against a predetermined fertility threshold, for example, the threshold (i.e., cut-off) level between individuals classified as infertile and sub-fertile, or the threshold level between individuals classified as sub-fertile and those classified as fertile.

In other embodiments, fertility thresholds may be determined by statistical analysis of the patterns found in sperm from a population of men, with known fertility. In an embodiment, a male is considered fertile or has normal male fertility if the male has a pregnant partner or has fathered a child within three years, using either natural conception or three or fewer cycles of intra-uterine insemination. In an embodiment, a male is considered sub-fertile if the male has failed to achieve a pregnancy with six to twelve months, without use of contraception, and required more than three cycles of intra-uterine insemination to achieve a pregnancy. In an embodiment, a male is considered infertile, if the male has failed to achieve a pregnancy within one year, without use of contraception, and failed to achieve a pregnancy using repeated cycles of intra-uterine insemination. In an embodiment, abnormal male fertility includes sub-fertile and infertile males.

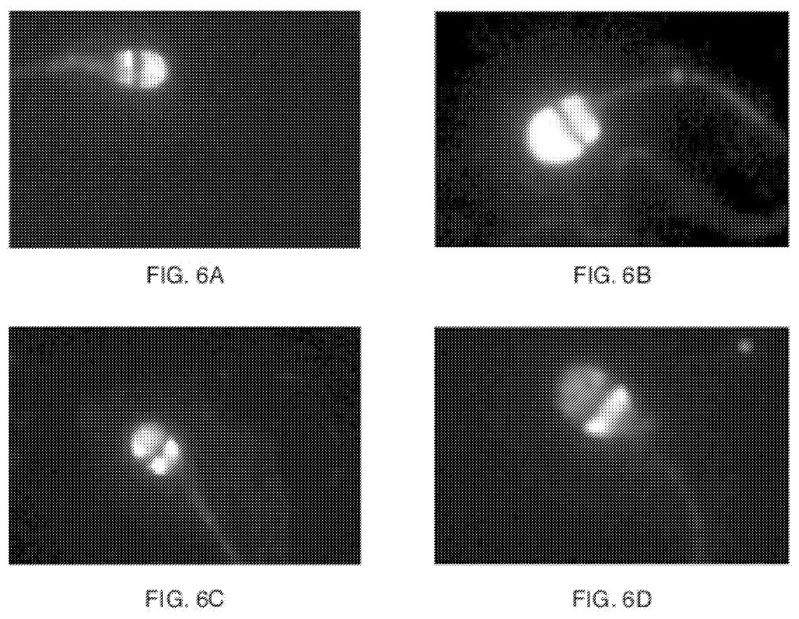

As shown in FIG. 4, 73 semen samples were obtained from 24 men known to be fertile. Their sperm was incubated with stimuli for capacitation, in this case 4 mM 2-hydroxy-propyl-βcyclodextrin, fixed with 0.01% paraformaldehyde (final concentration). The percentage of cells having patterns indicative of having capacitated (e.g., AA+APM) was assessed. The mean percentage of sperm having the AA and APM patterns was 41%, and two standard deviations from the mean was calculated as 27% and 55%.

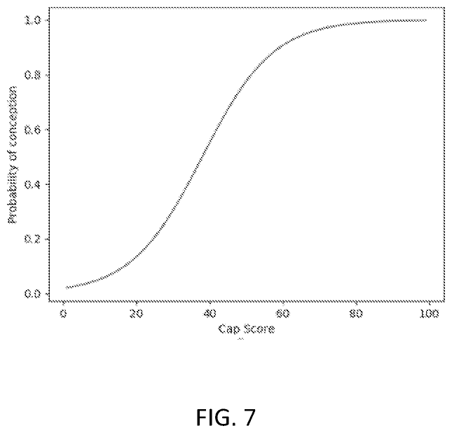

G_{M1} localization patterns in 14 samples from 14 men seeking medical evaluation of their fertility status were analyzed. The relative percentages of sperm having AA+APM localization patterns were compared against the statistical thresholds identified from the population of known fertile men (FIG. 5). There were no differences observed in the samples incubated under baseline (non-stimulating, non-capacitating conditions). However, 5 of the 14 men produced samples that showed low percentages of sperm with AA+APM patterns when incubated with 4 mM 2-hydroxy-propyl-β-cyclodextrin. These 5 samples all fell below two standard deviations from the mean. It is believed that approximately 30-50% of couples having difficulty conceiving have a component of male factor. These data fall within that expected range.

In one embodiment, the present disclosure provides kits for determination of male fertility status. The kit comprises one or more of the following: a pipette having an orifice of sufficient size in diameter to prevent shearing of a sperm membrane, agents that can act as stimuli for in vitro capacitation, capacitating media, non-capacitating media, fixative, labeling reagents s for determining of G_{M1} localization patterns, a diagram illustrating one or more G_{M1} localization patterns of capacitated sperm and one of more G_{M1} localization patterns of non-capacitated sperm. Such G_{M1} localization patterns of capacitated sperm and G_{M1} localization patterns of non-capacitated sperm are reflective of known fertility status. In such a kit embodiment, the fixative composition should not damage sperm membrane. In such embodiments, the reagent that can damage sperm membranes is chosen from the various reagents that are used to decrease semen viscosity. In some embodiments, the membrane damaging reagent includes one or more of a protease, a nuclease, a mucolytic agent, a lipase, an esterase and glycoside hydrolases. In another kit embodiment, the capacitating media and non-capacitating media, when applied in vitro to sperm cells, produce G_{M1} localization patterns indicative of capacitated sperm and patterns indicative of non-capacitated sperm as reflected in the diagram.

In one embodiment, the kit comprises an agent having 4% cyclodextrin to stimulate capacitation.

In one embodiment, the capacitating media comprises: modified human tubal fluid with added 2-hydroxy-propyl-β-cyclodextrin so as to give a 3 mM final concentration; the non-capacitating media comprises modified human tubal fluid; the fixative is 1% paraformaldehyde; and the reagent for determining G_{M1} patterns is cholera toxin's b subunit (15 μg/ml final concentration). In other embodiments, the final concentration of paraformaldehyde is 0.01%.

An exemplary kit comprises modified HTF medium with gentamicin buffered with HEPES (Irvine Scientific, reference 90126). No difference in G_{M1} localization patterns, viability or sperm recovery, and capacitation was observed whether bicarbonate- or HEPES-buffered medium was used. Therefore, bicarbonate buffered media can also be used. Use of the HEPES-buffer enables the assay to be performed in clinics using air incubators or water baths, as opposed to only being compatible with CO2 incubators. Similarly, adding supplemental proteins, whether commercial (HTF-SSS™, Irvine Scientific, or plasmanate), or powdered albumin did not alter recovery or viability, and favorably enhance capacitation status.

The exemplary kit can further comprise cell isolation media (such as Enhance S-Plus Cell Isolation Media, 90% from Vitrolife, reference: 15232 ESP-100-90%). The exemplary reagents, consumables and procedures were demonstrated to yield advantageous labeling of G_{M1} on human sperm.

The exemplary kit can further comprise wide orifice pipette tips (200 μl large orifice tip, USA scientific, 1011-8400). The exemplary kit can further comprise wide orifice transfer pipettes (General Purpose Transfer Pipettes, Standard Bulb reference number: 202-20S. VWR catalog number 14670-147). In one embodiment, the pipette is non-metallic. In some embodiments the wide orifice pipette has a gauge size of at least 18 gauge, 16 gauge or 14 gauge. In some embodiments, the wide orifice pipette has an orifice size of at least 1 mm, 1.2 mm or 1.4 mm.

The exemplary kit can further comprise 1.5 ml tubes (Treatment cap, noncap, CD) (USA Scientific 14159700)—one containing cyclodextrin in powdered form to stimulate capacitation, and one empty for noncapacitating conditions of media alone. In some embodiments, it is possible that the cyclodextrin will be found in a separate tube, to which medium will be added to make a stock solution, that itself would be added to the capacitating tube.

When isolating sperm from seminal plasma it is common for human andrology labs to collect sperm using density gradients. The exemplary kit can further comprise density gradient materials and/or instructions to remove the seminal plasma off the density gradient and then to collect the pelleted sperm using a fresh transfer pipette.

The exemplary kit can further comprise the fixative (such as 0.1% PFA), and optionally comprises informational forms (such as patient requisition form), labels and containers/bags/pouches and the like useful for shipping, storage or regulatory purposes. For example, the kit can contain a foil pouch, a biohazard bag with absorbent for mailing patient sample, a re-sealable bag with absorbent, and a foam tube place holder.

The exemplary kit can further include instructions describing any of the methods disclosed herein.

In another aspect, a method for measuring the fertility of a male individual is provided. The G_{M1} localization assay can show whether sperm can capacitate, and therefore become competent to fertilize an egg. As described above, the assay may be scored as percentages of the morphologically normal sperm that have specific patterns of G_{M1} localization in the sperm head. The APM and AA patterns increase as sperm respond to stimuli for capacitation. Cut-offs can be used to distinguish the relative fertility of the ejaculates, separating the semen samples into groups based on male fertility (e.g., distinguishing fertile from sub-fertile from infertile men). However, because sperm number, motility, and morphology can also influence male fertility, the present disclosure provides methods for creating an index of male fertility (the “male fertility index” or “MFI”) that encompasses Cap-Score and one or more relevant semen parameters (e.g., number, motility, and morphology, etc.). Cap-Score (also referred to as G_{M1} score) is the number of one or more G_{M1} patterns. For example, a Cap-Score can be a number of one or more of Lined-Cell, INTER, AA, and APM. Different indices can be generated that emphasize specific semen parameters. For example, indexes according to the present disclosure include:
Cap-Score×% with progressive motility×absolute number;
Cap-Score×% morphologically normal sperm×absolute number;
Cap-Score×% total motility×absolute number×% morphologically normal sperm; and
other variations or combinations of Cap-Score and these parameters, or other specific parameters including those obtained using CASA (computer assisted sperm analysis), such as: VSL (velocity straight line); STR (straightness); LIN (Linearity); VCL (curvilinear velocity); VAP (velocity average path); % motility; duration of motility; LHA (lateral head amplitude); WOB (wobble); PROG (progression); and BCF (Beat cross number), etc. See, e.g., World Health Organization, “WHO Laboratory Manual for the Examination and Processing of Human Sperm,” (Fifth Ed. 2010).

The male fertility index may be embodied as a method for measuring the fertility status of a male individual. A sperm sample is obtained, wherein the sperm sample is from the individual being measured and wherein at least a portion of the sperm sample has been exposed to in vitro capacitating conditions, exposed to a fixative, and stained for GM₁, as described above. The values of one or more semen parameters are obtained for the sperm sample, such as, for example, the volume of the original sample from the individual, and/or the concentration, motility, and/or morphology of the sperm of the sample. An MFI is determined from the number of one or more G_{M1} patterns (e.g., the CAPScore™) and the one or more obtained semen parameter values. In the examples used herein, the Cap-Score™ is the percentage of one or more G_{M1} patterns under capacitating conditions at three hours, but other variants of Cap-Scores will be apparent in light of this disclosure (e.g., number at other time intervals, change in number of a G_{M1} pattern in capacitated from non-capacitated, etc.)

In one embodiment, a male fertility index score may be calculated for a sample of men according to the following equation: Male Fertility Index/Fertility Group=a+b₁*x₁+b₂*x₂+ . . . +bₘ*xₘ where a is a constant, b₁ through bₘ are regression coefficients and x₁ though xₘ are male fertility variables such as Cap-Score, motility, morphology, volume, and concentration. Discriminant function analysis may be used to determine which fertility variables discriminate between two or more naturally occurring groups. For example, to determine if an individual falls into a fertile, sub-fertile or in-fertile group, data would be collected for numerous fertility variables that describe sperm function and semen quality. A Discriminant Analysis may then be used to determine which variable(s) is/are the best predictors of fertility group and relatively how much each fertility variable should be weighted.

The male fertility index may be generated by a lab that reads the G_{M1} localization assay. The lab may obtain a sperm sample, and a semen analysis corresponding to the sperm sample, from one or more facility (e.g., fertility clinic, sperm bank, etc.). Semen analysis information can be included on a card included with a G_{M1} localization assay kit, sent electronically to the lab, and/or otherwise provided. In another exemplary embodiment, the lab obtains the Cap-Score of a sperm sample and also obtains the semen analysis information for the sperm sample. In one embodiment, the lab calculates the male fertility index based on the obtained Cap-Score and the obtained semen analysis data.

An exemplary method for identifying fertility status of a male comprises exposing sperm sample from the individual to in vitro non-capacitating and in vitro capacitating conditions. The sperm are fixed and a percentage of selected G_{M1} patterns in the fixed sperm is determined. The percentage for different G_{M1} patterns in sperm exposed to in vitro non-capacitating and in vitro capacitating conditions is compared. A change in the percentage of one or more selected G_{M1} patterns in sperm exposed to in vitro capacitating conditions over sperm exposed to in vitro non-capacitating conditions is indicative of the fertility status of the individual. The selected G_{M1} patterns can be Lined-Cell, INTER, AA and/or APM. In one embodiment, the fertility status of the individual is determined by calculating a ratio for a sum of the number of AA G_{M1} localization patterns and number of APM G_{M1} localization patterns over a sum of the total number of G_{M1} labeled localization patterns for the capacitated sperm.

An exemplary method for identifying fertility status of a male comprises exposing a sperm sample from the individual to in vitro capacitating conditions. The sperm are fixed and a percentage of selected G_{M1} patterns in the fixed sperm is determined. The percentage for different G_{M1} patterns is compared to the percentage from a control, wherein the control sperm sample has been exposed to the same in vitro capacitating conditions and same fixative. A change in the percentage of one or more selected G_{M1} patterns relative to the change in the control is indicative of different fertility status of the individual than the fertility status of the control. The G_{M1} patterns can be Lined-Cell, INTER, AA and/or APM. In one embodiment, the fertility status of the individual is determined by calculating a ratio for a sum of the number of AA G_{M1} localization patterns and number of APM G_{M1} localization patterns over a sum of the total number of G_{M1} labeled localization patterns for the capacitated sperm. In one embodiment, prior to the exposing steps, a semen sample is treated to decrease semen viscosity using a wide orifice pipette made of non-metallic material and using a reagent that does not damage sperm membrane chosen from the various reagents that are used to decrease semen viscosity. In some embodiments, the membrane damaging reagent is selected from the group consisting of (i) a protease; (ii) a nuclease (iii) a mucolytic agent; (iv) a lipase; (v) an esterase and (vi) glycoside hydrolases. In some embodiments the wide orifice pipette has a gauge size of at least 18 gauge, 16 gauge or 14 gauge. In some embodiments, the wide orifice pipette has an orifice size of at least 1 mm, 1.2 mm or 1.4 mm.

In the exemplary method, the control can be a sperm sample from an individual who is known to have normal fertility status or an individual who is known to have abnormal fertility status. The control can be a value obtained from a dataset comprising a plurality of individuals, for example, a dataset comprising at least 50 individuals.

An exemplary method for identifying fertility status of a male as infertile, sub-fertile, or fertile, comprises exposing a sperm sample from the individual to in vitro capacitating conditions. G_{M1} patterns in the sample are determined. The percentage of one or more G_{M1} patterns is compared to a fertility threshold wherein a percentage less than the fertility threshold is indicative of fertility problems. For example, a percentage less than the fertility threshold can be indicative of a fertility status of infertile or sub-fertile. The G_{M1} patterns can be Lined-Cell, INTER, AA and/or APM. In one embodiment, the fertility status of the individual is determined by calculating a ratio for a sum of the number of AA G_{M1} localization patterns and number of APM G_{M1} localization patterns over a sum of the total number of G_{M1} labeled localization patterns for the capacitated sperm. In one embodiment, prior to the exposing steps, a semen sample is treated to decrease semen viscosity using a wide orifice pipette made of non-metallic material and using a reagent that does not damage sperm membrane chosen from the various reagents that are used to decrease semen viscosity. In some embodiments, the membrane damaging reagent is selected from the group consisting of (i) a protease; (ii) a nuclease (iii) a mucolytic agent; (iv) a lipase; (v) an esterase and (vi) glycoside hydrolases. In some embodiments the wide orifice pipette has a gauge size of at least 18 gauge, 16 gauge or 14 gauge. In some embodiments, the wide orifice pipette has an orifice size of at least 1 mm, 1.2 mm or 1.4 mm.

The in vitro capacitating conditions in the exemplary methods can include exposure to i) bicarbonate and calcium ions, and ii) mediators of sterol efflux such as 2-hydroxy-propyl-β-cyclodextrin, methyl-β-cyclodextrin, serum albumin, high density lipoprotein, phospholipids vesicles, fetal cord serum ultrafiltrate, fatty acid binding proteins, or liposomes. In the exemplary methods, exposure of the control to capacitating or non-capacitating conditions can be done in parallel with the test sample.

An exemplary method for identifying fertility status of a male as infertile, sub-fertile, or fertile, comprises exposing a sperm sample from the individual to capacitating conditions. The percentage of each G_{M1} pattern in the sample is determined. The percentage of one or more G_{M1} patterns is compared to an infertility threshold wherein a percentage less than the infertility threshold is indicative of fertility problems. The capacitating conditions in the exemplary method can include exposure to i) bicarbonate and calcium ions, and ii) mediators of sterol efflux such as 2-hydroxy-propyl-β-cyclodextrin, methyl-β-cyclodextrin, serum albumin, high density lipoprotein, phospholipids vesicles, fetal cord serum ultrafiltrate, fatty acid binding proteins, or liposomes. The one or more G_{M1} localization patterns can be Lined-Cell, INTER, AA and/or APM. In one embodiment, the fertility status of the individual is determined by calculating a ratio for a sum of the number of AA G_{M1} localization patterns and number of APM G_{M1} localization patterns over a sum of the total number of G_{M1} labeled localization patterns for the capacitated sperm. In one embodiment, prior to the exposing steps, a semen sample is treated to decrease semen viscosity using a wide orifice pipette made of non-metallic material and using a reagent that does not damage sperm membrane chosen from the various reagents that are used to decrease semen viscosity. In some embodiments, the membrane damaging reagent is selected from the group consisting of (i) a protease; (ii) a nuclease (iii) a mucolytic agent; (iv) a lipase; (v) an esterase and (vi) glycoside hydrolases. In some embodiments the wide orifice pipette has a gauge size of at least 18 gauge, 16 gauge or 14 gauge. In some embodiments, the wide orifice pipette has an orifice size of at least 1 mm, 1.2 mm or 1.4 mm.

The fertility threshold in the exemplary methods can be the AA+APM pattern percentage at which the fertility of a population ceases to substantially increase. For example, the fertility threshold can be a level of AA+APM at which more than 50% of the population are fertile; a level of AA+APM at which more than 60-85% of a population is fertile; or a level of AA+APM in the range of 35-40 (relative percentage of total G_{M1} patterns), inclusive. The fertility threshold can be 38, 38.5, 39, or 39.5% AA+APM (relative to total G_{M1} patterns).

An exemplary method may further comprise comparing the percentage of one or more G_{M1} patterns to an infertility threshold wherein a percentage less than the infertility threshold is indicative of infertility. For example, the infertility threshold can be the AA+APM pattern percentage at which the fertility of a population begins to substantially increase; a level of AA+APM at which less than 50% of the population are fertile; a level of AA+APM at which more than 60-85% of a population is fertile; or a level of AA+APM in the range of 14-18 (relative percentage of total G_{M1} patterns), inclusive. The infertility threshold can be 14, 14.5, 15, or 15.5% AA+APM (relative to total G_{M1} patterns).

An exemplary method for identifying fertility status of a male comprises obtaining sperm samples, wherein the sperm samples are from the individual and wherein the sperm samples have been exposed to non-capacitating or capacitating conditions, fixed, and stained for G_{M1}. The number of selected G_{M1} patterns in the sperm is determined. The percentage for different G_{M1} patterns in sperm exposed to in vitro non-capacitating and in vitro capacitating conditions is compared. A change in the percentage of one or more selected G_{M1} patterns in sperm exposed to in vitro capacitating conditions over sperm exposed to in vitro non-capacitating conditions is indicative of the fertility status of the individual. The G_{M1} pattern can be selected from the group consisting of AA, APM, INTER, Lined-Cell and combinations thereof. In one embodiment, the fertility status of the individual is determined by calculating a ratio for a sum of the number of AA G_{M1} localization patterns and number of APM G_{M1} localization patterns over a sum of the total number of G_{M1} labeled localization patterns for the capacitated sperm.

An exemplary method for identifying fertility status of a male individual comprises obtaining a sperm sample, wherein the sperm sample is from the individual and wherein the sperm sample has been exposed to in vitro capacitating conditions, has been fixed and has been stained for the presence of G_{M1}. A number of selected G_{M1} patterns in the sperm is determined. The percentage for one or more different G_{M1} patterns is compared to the percentage of patterns from a control or predetermined criteria. The control sperm sample has been exposed to the same in vitro capacitating conditions and same fixative. A change in the percentage of one or more selected G_{M1} patterns relative to the change in the control is indicative of different fertility status of the individual than the fertility status of the control.

An exemplary method for identifying fertility status of a male individual comprises obtaining a sperm sample, wherein the sperm sample is from the individual, and wherein the sperm sample has been exposed to in vitro capacitating conditions, has been fixed, and has been stained for G_{M1} patterns. The G_{M1} localization patterns in the sample are determined. The percentage of one or more G_{M1} patterns is compared to an infertility threshold wherein a percentage less than the infertility threshold is indicative of fertility problems.

An exemplary method for measuring the fertility status of a male individual comprises obtaining a sperm sample, wherein the sperm sample is from the individual, and wherein the sperm sample has been exposed to in vitro capacitating conditions, has been exposed to a fixative, and has been stained for G_{M1}. Values are obtained for one or more of volume of the original sample, and concentration, motility, and morphology of the sperm in the original sample. A Cap-Score of the sperm sample is determined as the percentage of one or more G_{M1} localization patterns in the sample. A male fertility index (WI) value of the individual is calculated based on the determined Cap-Score and the one or more obtained volume, concentration, motility, and morphology. For example, the MFI value can be calculated by multiplying the Cap-Score™, the volume, the concentration, the motility value, and the morphology value. The motility can be a percentage of the sperm which are motile. The morphology can be a percentage of the sperm that are morphologically normal.

An exemplary method for measuring the fertility status of a male individual comprises obtaining a Cap-Score™ of a sperm sample of the individual as the percentage of one or more G_{M1} localization patterns in the sample. Values are obtained for one or more of volume of the original sample, and concentration, motility, and morphology of the sperm in the original sample. A male fertility index (MFI) value of the individual is calculated based on the determined Cap-Score and the one or more obtained volume, concentration, motility, and morphology.

### Regression and Computer Learning Modeling

In some aspects, the present disclosure provides methods, systems, and computer readable medium (e.g., non-transitory computer readable medium) for characterizing the fertility status of a male (e.g., predicting a probability that use of the male's sperm will generate a pregnancy, (PGP), for example under natural conditions or under an assisted reproduction method, such as intra-uterine insemination (IUI)), using one or more of a broad array of classification methods known to those of skill in the art. In some aspects, the present disclosure also provides methods, systems, and computer readable medium (e.g., non-transitory computer readable medium) for training a fertility classifier for characterizing the fertility status of a male (e.g., predicting a probability of generating a pregnancy, for example under natural conditions or under an assisted reproduction method, such as intra-uterine insemination (IUI), will result in pregnancy).

### Classification Methods

In some embodiments a model 214 is trained using machine learning techniques or methods. Machine learning methods allow a computer system to perform automatic (e.g., through software programs) learning from a set of factual data (e.g., training sets of features from semen samples of males of couples who have attempted to become pregnant using assisted reproduction methods), belonging to a specific application field (e.g., domain). Given such a training set, machine learning methods are able to extract patterns and relationships from the data themselves. An extensive discussion about machine learning methods and their applications can be found in Mitchell, 1997, Machine Learning, McGraw-Hill and U.S. Pat. No. 8,843,482, each of which is hereby incorporated by reference. Well-known machine learning methods include decision trees, association rules, neural networks, and Bayesian methods.

Learned patterns and relationships are encoded by machine learning methods in a formal, quantitative model, which can take different forms depending on the machine learning technique used. Examples of forms for a model include logic rules, mathematical equations, and mathematical graphs. A goal of machine learning methods is that of a better understanding and quantification of patterns within data and relationships between data in order to obtain a model as a representation for the data, e.g., a model for representing male fertility.

In some embodiments the model is trained against a single feature across the training set (e.g., whether or not pregnancy was achieved using an assisted reproduction method, such as IUI). In some embodiments this single second feature is categorical (e.g., pregnant or not pregnant). In some embodiments this single second feature is numerical (e.g., the number of rounds of assisted reproduction performed prior to pregnancy). In some embodiments, the model is trained against a combination of single features across the training set. In some embodiments values for second features in the training set are not used to train the model. In some embodiments, kernel transformation techniques and/or principal component analysis techniques are used to identify the set of first features (e.g., parameters {β₀, β₁, . . . , βᵢ}) as disclosed with respect to some detailed embodiments below. As such, it will be appreciated that, in some embodiments, the set of first features {β₀, β₁, . . . , βᵢ} is in the form of principal components and it is the principal components that are used to train any of the male fertility models described herein. In other embodiments, the measurements of the set of first features {β₀, β₁, . . . , βᵢ} themselves, not in the form of principal components, are used to train any of the models described herein.

In some embodiments, the male fertility model is a supervised regression model and the trained model provides predictions of real values for a single second feature (e.g., a prediction of how many rounds of assisted reproduction will be needed before achieving pregnancy). Such approaches are useful instances where the target second feature (e.g., time to pregnancy) is measured as a continuous number.

In some embodiments, the male fertility model is a supervised classification model and the trained model provides a prediction of a classification for a single second feature (e.g., a prediction as to the chance of a couple becoming pregnant using an assisted reproduction technique). Such approaches are useful instances where the target second feature (e.g., pregnancy) is measured as a discrete label.

In some embodiments, the model 214 is an unsupervised clustering model or a nearest neighbor search model. In such an unsupervised approach, models quantify overall correspondence among reference entities.

In some embodiments, an ensemble (two or more) of models is used. In some embodiments, a boosting technique such as AdaBoost is used in conjunction with many other types of learning algorithms to improve their performance. In this approach, the output of any of the models disclosed herein, or their equivalents, is combined into a weighted sum that represents the final output of the boosted classifier. See Freund, 1997, “A decision-theoretic generalization of on-line learning and an application to boosting,” Journal of Computer and System Sciences 55, p. 119, which is hereby incorporated by reference.

In some embodiments, the trained male fertility model is a nonlinear regression model. In nonlinear regression approaches, each Xⱼ in {X₁, . . . , Xᵢ} is modeled as a random variable with a mean given by a nonlinear function ƒ(x,β). See Seber and Wild, 1989, Nonlinear Regression, New York: John Wiley and Sons, ISBN 0471617601, which is hereby incorporated by reference.

In one embodiment, the trained male fertility model is a logistic regression model, e.g., of the form:

$$f ( X ) = \frac{1}{1 + exp ( - ( {β}_{0} + {\sum }_{j = 1}^{i} {β}_{j} {X}_{j} ) )}$$

where ƒ(X) is a measure of fertility, i is a positive integer, α is parameter determined during training of the pre-trained classifier, β₀, β₁, . . . , βᵢ are parameters determined during training of the pre-trained classifier, and each Xⱼ in {X₁, . . . , Xᵢ} is a datum in the data obtained from the sperm sample (e.g., including one or more of a ratio between (i) a combination of the AA GM1 localization pattern and APM GM1 localization patterns and (ii) a combination of all the GM1 labeled localization patterns, a volume of the sperm sample, a concentration of sperm in the sperm sample, a motility of sperm in the sperm sample, an interaction term thereof, a transformation of a datum thereof, a basis expansion of a datum thereof, and a principle component expressed as a linear component of two or more data thereof.). See, Hastie et al., 2001, The Elements of Statistical Learning, pp. 42-49; and Jolliffe, 1982, “A note on the Use of Principal Components in Regression,” Journal of the Royal Statistical Society, Series C. 31 (3), pp. 300-303, each of which is hereby incorporated by reference.

Examples of a transformation of a first datum include, but are not limited to, a log, square-root, a square, or, in general, raising the value of the datum to a power. An example of a basis expansion of the datum include, but are not limited to representing the datum as a polynomial, a piecewise polynomial or a smoothing spline as discussed in Hastie et al., 2001, The Elements of Statistical Learning, Chapter 5, which is hereby incorporated by reference. An example of an interaction between two or more datum is X₁·X₂.

In some embodiments, the trained male fertility classification model is a linear regression model of the form:

ƒ(X)=β₀+Σⱼ₌₁^{t}Xⱼβⱼ

where t is a positive integer, f(X) is a measure of male fertility, β₀, β₁, . . . , βₜ are parameters that are determined by the training of the model, and each Xᵢ in {X₁, . . . , Xₜ} is a datum in the data obtained from the sperm sample (e.g., including one or more of a ratio between (i) a combination of the AA GM1 localization pattern and APM GM1 localization patterns and (ii) a combination of all the GM1 labeled localization patterns, a volume of the sperm sample, a concentration of sperm in the sperm sample, a motility of sperm in the sperm sample, an interaction term thereof, a transformation of a datum thereof, a basis expansion of a datum thereof, and a principle component expressed as a linear component of two or more data thereof). See, Hastie et al., 2001, The Elements of Statistical Learning, pp. 42-49; and Jolliffe, 1982, “A note on the Use of Principal Components in Regression,” Journal of the Royal Statistical Society, Series C. 31 (3), pp. 300-303, each of which is hereby incorporated by reference.

In some embodiments, the trained male fertility classification model is a support vector machine (SVM). In such embodiments, SVMs are trained to classify a respective entity using measurements of the sperm sample data {X₁, . . . , Xᵢ} across a training set and a measurement of an outcome (e.g., pregnancy and/or time to pregnancy) across the training set. SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data training set (e.g., the target outcome is provided with a binary label of either possessing the target outcome (e.g., pregnancy) or not possessing the target outcome (e.g., failure to become pregnant) with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space.

In some embodiments, the trained male fertility classification model is a principal component analysis (PCA) model. PCA can be used to analyze sperm sample characteristic data of the training set in order to construct a decision rule that discriminates a label (e.g., pregnancy or non-pregnancy). PCA reduces the dimensionality of the training set 206 by transforming the sperm sample characteristic data of the training set to a new set of variables (principal components) that summarize the features of the training set. See, for example, Jolliffe, 1986, Principal Component Analysis, Springer, New York, which is hereby incorporated by reference. PCA is also described in Draghici, 2003, Data Analysis Tools for DNA Microarrays, Chapman & Hall/CRC, which is hereby incorporated by reference.

Principal components (PCs) are uncorrelated and are ordered such that the kth PC has the kth largest variance among PCs. The kth PC can be interpreted as the direction that maximizes the variation of the projections of the data points such that it is orthogonal to the first k−1 PCs. The first few PCs capture most of the variation in the training set. In contrast, the last few PCs are often assumed to capture only the residual ‘noise’ in the training set. As such, PCA can also be used to create a model in accordance with the present disclosure. In such an approach, each row in a table is constructed and represents the measurements for the sperm sample characteristic data from a particular reference entity of the training set and can be considered a vector. As such, the data in the training set can be viewed as matrix of vectors, each vector representing a respective reference entity and including measurements for sperm sample characteristic data from respective males in the training set. In some embodiments, this matrix is represented in a Free-Wilson method of qualitative binary description of monomers (Kubinyi, 1990, 3D QSAR in drug design theory methods and applications, Pergamon Press, Oxford, pp 589-638), and distributed in a maximally compressed space using PCA so that the first principal component (PC) captures the largest amount of variance information possible, the second principal component (PC) captures the second largest amount of all variance information, and so forth until all variance information in the matrix has been considered. Then, each of the vectors (where each vector represents a reference entity of the training set) is plotted.

### Feature Selection Methods

In some embodiments, the fertility classification methods used and/or trained, as described herein, are based on features of sperm samples that are selected using a feature selection method, e.g., a least angle regression or a stepwise regression. Feature selection methods are particularly advantageous in identifying, from among the multitude of variables (e.g., Cap-Score, sperm number (e.g., concentration), sperm motility, sperm morphology, other sperm movement metrics, such as VSL (velocity straight line), STR (straightness), LIN (Linearity), VCL (curvilinear velocity), VAP (velocity average path), % motility, duration of motility, LHA (lateral head amplitude), WOB (wobble), PROG (progression), and BCF (Beat cross number), and other biometric data from the male subject, such as age, weight, etc.) present across the training set, which features have a significant causal effect on a given outcome (e.g., which sperm characteristics are causal for a low male fertility and/or a high male fertility). Feature selection techniques are described, for example, in Saeys et al., 2007, “A Review of Feature Selection Techniques in Bioinformatics,” Bioinformatics 23, 2507-2517, and Tibshirani, 1996, “Regression and Shrinkage and Selection via the Lasso,” J. R. Statist. Soc. B, pp. 267-288, each of which is hereby incorporated by reference.

In some embodiments, the feature selection method includes regularization (e.g., is Lasso, least-angle-regression, or Elastic net) across the training set to improve prediction accuracy. Lasso is described in Hastie et al., 2001, The Elements of Statistical Learning, pp. 64-65, which is hereby incorporated by reference. Least angle regression is described in Efron et al., 2004, “Least Angle Regression,” The Annals of Statistics, pp. 407-499, which is hereby incorporated by reference. Elastic net, which encompasses ridge regression, is described in Hastie, 2005, “Regularization and Variable Selection via the Elastic Net,” Journal of the Royal Statistical Society, Series B: pp. 301-320, which is hereby incorporated by reference.

In some embodiments, the feature selection method comprises application of a decision tree to the training set. Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

Another feature selection method that can be used in the system and methods of the present disclosure is multivariate adaptive regression splines (MARS). MARS is an adaptive procedure for regression, and is well suited for the high-dimensional problems addressed by the present disclosure. MARS can be viewed as a generalization of stepwise linear regression or a modification of the CART method to improve the performance of CART in the regression setting. MARS is described in Hastie et al., 2001, The Elements of Statistical Learning, Springer-Verlag, New York, pp. 283-295, which is hereby incorporated by reference in its entirety.

In some embodiments, the feature selection method comprises application of Gaussian process regression to the training set. Gaussian Process Regression is disclosed in Ebden, August 2008, arXiv:1505.029652v2 (29 Aug. 2015), “Gaussian Processes for Dimensionality Reduction: A Quick Introduction,” which is hereby incorporated by reference.

Exemplary Classification Methods, Systems, and Computer Readable Medium

Now that an overview of different classification models and feature selection models that are used in various embodiments of the present disclosure have been outlined, more details of specific models and model training are provided.

In one aspect, the disclosure provides a method for characterizing a fertility status of a male comprising: exposing, in vitro, a portion of a sperm sample from a male to capacitating conditions, thereby forming a capacitated sperm sample, fixing the capacitated sperm sample with a fixative, thereby forming a fixed in vitro capacitated sperm sample, treating the fixed in vitro capacitated sperm sample with a labeling molecule for G_{M1} localization patterns, wherein the labeling molecule has a detectable label, thereby forming a labeled fixed in vitro capacitated sperm sample, identifying a plurality of G_{M1} labeled localization patterns for the labeled fixed in vitro capacitated sperm sample, said plurality of G_{M1} labeled localization patterns comprising an apical acrosome (AA) G_{M1} localization pattern, an acrosomal plasma membrane (APM) G_{M1} localization pattern, a Lined-Cell G_{M1} localization pattern and all other labeled G_{M1} localization patterns, assigning the AA G_{M1} localization pattern and the APM G_{M1} localization pattern to a capacitated state, assigning the Lined-Cell G_{M1} localization pattern and all other labeled G_{M1} localization patterns to a non-capacitated state, and characterizing a fertility status of the male by applying one or more pre-trained fertility classifiers to data obtained from the sperm sample, wherein the data obtained from the sperm sample comprises a ratio between (i) a combination of the AA G_{M1} localization pattern and APM G_{M1} localization patterns and (ii) a combination of all the G_{M1} labeled localization patterns (e.g., a ratio of sperm displaying a capacitated state to a total number of assigned sperm).

In some embodiments, the data obtained from the sperm sample consists of the ratio between (i) the combination of the AA G_{M1} localization pattern and APM G_{M1} localization pattern and (ii) the combination of all the G_{M1} labeled localization patterns. In some embodiments, the data obtained from the sperm sample further comprises one or more datum selected from the group consisting of (A) a volume of the sperm sample, (B) a concentration of sperm in the sperm sample, (C) a motility of sperm in the sperm sample, and (D) an arithmetic combination of any two of (e.g., an interaction term): (a) the ratio between (i) the combination of the AA G_{M1} localization pattern and APM G_{M1} localization pattern and (ii) the combination of all the G_{M1} labeled localization patterns, (b) the volume of the sperm sample, (c) the concentration of sperm in the sperm sample, and (d) the motility of sperm in the sperm sample. In some embodiments, the arithmetic combination is a sum, a difference, a product, or a ratio of any two data measures. In some embodiments, the data obtained from the sperm sample consists of: (A) the ratio between (i) the combination of the AA G_{M1} localization pattern and APM G_{M1} localization pattern and (ii) the combination of all the G_{M1} labeled localization patterns, (B) the volume of the sperm sample, and (C) a product of (a) the ratio between (i) the combination of the AA G_{M1} localization pattern and APM G_{M1} localization pattern and (ii) the combination of all the G_{M1} labeled localization patterns, and (b) the volume of the sperm sample.

In some embodiments, a classifier in the one or more pre-trained fertility classifiers is a nonlinear regression model. In some embodiments, a classifier in the one or more pre-trained fertility classifiers is a logistic regression model, e.g., of the form:

$$f ( X ) = \frac{1}{1 + exp ( - ( {β}_{0} + {\sum }_{j = 1}^{i} {β}_{j} {X}_{j} ) )}$$

wherein: ƒ(X) is a measure of fertility, i is a positive integer, α is parameter determined during training of the pre-trained classifier, β₀, β₁, . . . , βᵢ are parameters determined during training of the pre-trained classifier, and each Xⱼ in {X₁, . . . , Xᵢ} is a datum in the data obtained from the sperm sample (e.g., including one or more of a ratio between (i) a combination of the AA GM1 localization pattern and APM GM1 localization patterns and (ii) a combination of all the GM1 labeled localization patterns, a volume of the sperm sample, a concentration of sperm in the sperm sample, a motility of sperm in the sperm sample, and an interaction term thereof).

In some embodiments, the capacitating conditions include exposure of the portion of the sperm sample to one or more of bicarbonate ions, calcium ions, and a mediator of sterol efflux. In some embodiments, the mediator of sterol efflux comprises 2-hydroxy-propyl-β-cyclodextrin, methyl-β-cyclodextrin, serum albumin, high density lipoprotein, phospholipid vesicles, fetal cord serum ultrafiltrate, fatty acid binding proteins, or liposomes. In some embodiments, the mediator of sterol efflux comprises 2-hydroxy-propyl-β-cyclodextrin.

In some embodiments, the fixative comprises paraformaldehyde, glutaraldehyde or a combination thereof.

In some embodiments, the labeling molecule for G_{M1} localization patterns comprises a fluorescently-labeled cholera toxin b subunit.

In some embodiments, the identifying step is performed from 2 to 24 hours after the exposing step.

In some embodiments, the method further includes the step of: prior to the exposing step, treating the portion of the sperm sample to decrease the viscosity of the portion of the sperm sample using a wide orifice pipette made of non-metallic material and using a reagent that does not damage sperm membranes.

In one aspect, the present disclosure provides a method comprising: obtaining a first portion of a portion of a sperm sample from a male that has been exposed to in vitro capacitating conditions, fixed in a fixative, and stained with a labeling molecule for G_{M1} localization patterns, wherein the labeling molecule has a detectable label, identifying a plurality of G_{M1} labeled localization patterns for the labeled fixed in vitro capacitated sperm sample, said plurality of G_{M1} localization patterns comprising an apical acrosome (AA) G_{M1} localization pattern, an acrosomal plasma membrane (APM) G_{M1} localization pattern, a Lined-Cell G_{M1} localization pattern and all other labeled G_{M1} localization patterns, assigning the AA G_{M1} localization pattern and the APM G_{M1} localization pattern to a capacitated state, assigning the Lined-Cell G_{M1} localization pattern and all other labeled G_{M1} localization patterns to a non-capacitated state, and characterizing a fertility status of the male by applying one or more pre-trained fertility classifiers to data obtained from the sperm sample, wherein the data obtained from the sperm sample comprises a ratio between (i) a combination of the AA G_{M1} localization pattern and APM G_{M1} localization pattern and (ii) a combination of the G_{M1} labeled localization patterns (e.g., a ratio of sperm displaying a capacitated state to a total number of assigned sperm).

In some embodiments, the data obtained from the sperm sample consists of the ratio between (i) the combination of the AA G_{M1} localization pattern and APM G_{M1} localization pattern and (ii) the combination of all the G_{M1} labeled localization patterns. In some embodiments, the data obtained from the sperm sample further comprises one or more datum selected from the group consisting of: (A) a volume of the sperm sample, (B) a concentration of sperm in the sperm sample, (C) a motility of sperm in the sperm sample, and (D) an arithmetic combination of any two of (a) the ratio between (i) the combination of the AA G_{M1} localization pattern and APM G_{M1} localization pattern and (ii) the combination of all the G_{M1} labeled localization patterns, (b) the volume of the sperm sample, (c) the concentration of sperm in the sperm sample, and (d) the motility of sperm in the sperm sample. In some embodiments, the arithmetic combination is a sum, a difference, a product, or a ratio of any two data measures. In some embodiments, the data obtained from the sperm sample consists of: (A) the ratio between (i) the combination of the AA G_{M1} localization pattern and APM G_{M1} localization pattern and (ii) the combination of all the G_{M1} labeled localization patterns, (B) the volume of the sperm sample, and (C) a product of (a) the ratio between (i) the combination of the AA G_{M1} localization pattern and APM G_{M1} localization pattern and (ii) the combination of all the G_{M1} labeled localization patterns, and (b) the volume of the sperm sample.

In some embodiments, a classifier in the one or more pre-trained fertility classifiers is a nonlinear regression model. In some embodiments, a classifier in the one or more pre-trained fertility classifiers is a logistic regression model, e.g., of the form:

$$f ( X ) = \frac{1}{1 + exp ( - ( {β}_{0} + {\sum }_{j = 1}^{i} {β}_{j} {X}_{j} ) )}$$

wherein: ƒ(X) is a measure of fertility, i is a positive integer, α is parameter determined during training of the pre-trained classifier, β₀, β₁, . . . , βᵢ are parameters determined during training of the pre-trained classifier, and each Xⱼ in {X₁, . . . , Xᵢ} is a datum in the data obtained from the sperm sample (e.g., including one or more of a ratio between (i) a combination of the AA GM1 localization pattern and APM GM1 localization patterns and (ii) a combination of all the GM1 labeled localization patterns, a volume of the sperm sample, a concentration of sperm in the sperm sample, a motility of sperm in the sperm sample, and an interaction term thereof).

In some embodiments, the capacitating conditions include exposure of the portion of the sperm sample to one or more of bicarbonate ions, calcium ions, and a mediator of sterol efflux. In some embodiments, the mediator of sterol efflux comprises 2-hydroxy-propyl-β-cyclodextrin, methyl-β-cyclodextrin, serum albumin, high density lipoprotein, phospholipid vesicles, fetal cord serum ultrafiltrate, fatty acid binding proteins, or liposomes. In some embodiments, the mediator of sterol efflux comprises 2-hydroxy-propyl-β-cyclodextrin.

In some embodiments, the fixative comprises paraformaldehyde, glutaraldehyde or a combination thereof.

In some embodiments, the labeling molecule for G_{M1} localization patterns comprises a fluorescently-labeled cholera toxin b subunit.

In some embodiments, the identifying step is performed from 2 to 24 hours after the exposing step.

In some embodiments, the method further includes, prior to the obtaining step, treating the portion of the sperm sample to decrease the viscosity of the portion of the sperm sample using a wide orifice pipette made of non-metallic material and using a reagent that does not damage sperm membranes.

In one aspect, the disclosure provides a method comprising the steps of: obtaining a sperm sample, wherein at least a portion of the sperm sample has been exposed to in vitro capacitating conditions to obtain an in vitro capacitated sperm, that been exposed to a fixative, and has been stained for G_{M1}, thereby forming a labeled fixed in vitro capacitated sperm sample, determining a Cap-Score of the labeled fixed in vitro capacitated sperm sample based on one or more G_{M1} labeled localization patterns, said G_{M1} labeled localization patterns being an apical acrosome (AA) G_{M1} localization pattern, a post-acrosomal plasma membrane (APM) G_{M1} localization pattern, a Lined-Cell G_{M1} localization pattern and all other labeled G_{M1} localization patterns, and characterizing a fertility status of the male by applying one or more pre-trained fertility classifiers to data obtained from the sperm sample, wherein the data comprises a ratio between (i) a combination of the AA G_{M1} localization pattern and the APM G_{M1} localization pattern and (ii) a combination of all the G_{M1} labeled localization patterns (e.g., a ratio of sperm displaying a capacitated state to a total number of assigned sperm).

In some embodiments, the data obtained from the sperm sample consists of the ratio between (i) the combination of the AA G_{M1} localization pattern and APM G_{M1} localization pattern and (ii) the combination of all the G_{M1} labeled localization patterns. In some embodiments, the data obtained from the sperm sample further comprises one or more datum selected from the group consisting of: (A) a volume of the sperm sample, (B) a concentration of sperm in the sperm sample, (C) a motility of sperm in the sperm sample, and (D) an arithmetic combination of any two of (a) the ratio between (i) the combination of the AA G_{M1} localization pattern and APM G_{M1} localization pattern and (ii) the combination of all the G_{M1} labeled localization patterns, (b) the volume of the sperm sample, (c) the concentration of sperm in the sperm sample, and (d) the motility of sperm in the sperm sample. In some embodiments, the arithmetic combination is a sum, a difference, a product, or a ratio of any two data measures. In some embodiments, the data obtained from the sperm sample consists of: (A) the ratio between (i) the combination of the AA G_{M1} localization pattern and APM G_{M1} localization pattern and (ii) the combination of all the G_{M1} labeled localization patterns, (B) the volume of the sperm sample, and (C) an product of (a) the ratio between (i) the combination of the AA G_{M1} localization pattern and APM G_{M1} localization pattern and (ii) the combination of all the G_{M1} labeled localization patterns, and (b) the volume of the sperm sample.

In some embodiments, a classifier in the one or more pre-trained fertility classifiers is a nonlinear regression model. In some embodiments, a classifier in the one or more pre-trained fertility classifiers is a logistic regression model, e.g., of the form:

$$f ( X ) = \frac{1}{1 + exp ( - ( {β}_{0} + {\sum }_{j = 1}^{i} {β}_{j} {X}_{j} ) )}$$

wherein: ƒ(X) is a measure of fertility, i is a positive integer, α is parameter determined during training of the pre-trained classifier, β₀, β₁, . . . , βᵢ are parameters determined during training of the pre-trained classifier, and each Xⱼ in {X₁, . . . , Xᵢ} is a datum in the data obtained from the sperm sample (e.g., including one or more of a ratio between (i) a combination of the AA GM1 localization pattern and APM GM1 localization patterns and (ii) a combination of all the GM1 labeled localization patterns, a volume of the sperm sample, a concentration of sperm in the sperm sample, a motility of sperm in the sperm sample, and an interaction term thereof).

In some embodiments, the method further includes the step of: prior to the obtaining step, treating the portion of the sperm sample to decrease the viscosity of the portion of the sperm sample using a wide orifice pipette made of non-metallic material and using a reagent that does not damage sperm membranes.

In one aspect, the present disclosure provides a method, comprising: characterizing a fertility status of a male by applying one or more pre-trained fertility classifiers to data obtained from a sperm sample from the male, wherein the data comprises a ratio between (i) a combination of apical acrosome (AA) G_{M1} localization patterns and acrosomal plasma membrane (APM) G_{M1} localization patterns and (ii) a combination all G_{M1} labeled localization patterns in a treated portion of the sperm sample, wherein the ratio between (i) the combination of the AA GM1 localization patterns and APM GM1 localization patterns and (ii) the combination of all G_{M1} labeled localization patterns is determined by: exposing, in vitro, a portion of the sperm sample from the male to capacitating conditions, thereby forming a capacitated sperm sample, fixing the capacitated sperm sample with a fixative, thereby forming a fixed in vitro capacitated sperm sample, treating the fixed in vitro capacitated sperm sample with a labeling molecule for G_{M1} localization patterns, wherein the labeling molecule has a detectable label, thereby forming a labeled fixed in vitro capacitated sperm sample, identifying a plurality of G_{M1} labeled localization patterns for the labeled fixed in vitro capacitated sperm sample, said plurality of G_{M1} labeled localization patterns comprising an AA G_{M1} localization pattern, an APM G_{M1} localization pattern, a Lined-Cell G_{M1} localization pattern and all other labeled G_{M1} localization patterns, assigning the AA G_{M1} localization pattern and the APM G_{M1} localization pattern to a capacitated state, assigning the Lined-Cell G_{M1} localization pattern and all other labeled G_{M1} localization patterns to a non-capacitated state, and comparing (i) the combination of the AA G_{M1} localization pattern and APM G_{M1} localization pattern to (ii) the combination of all the G_{M1} labeled localization patterns.

In some embodiments, the data obtained from the sperm sample consists of the ratio between (i) the combination of the AA G_{M1} localization pattern and APM G_{M1} localization pattern and (ii) the combination of all the G_{M1} labeled localization patterns. In some embodiments, the data obtained from the sperm sample further comprises one or more datum selected from the group consisting of: (A) a volume of the sperm sample, (B) a concentration of sperm in the sperm sample, (C) a motility of sperm in the sperm sample, and (D) an arithmetic combination of any two of (a) the ratio between (i) the combination of the AA G_{M1} localization pattern and APM G_{M1} localization pattern and (ii) the combination of all the G_{M1} labeled localization patterns, (b) the volume of the sperm sample, (c) the concentration of sperm in the sperm sample, and (d) the motility of sperm in the sperm sample. In some embodiments, the data obtained from the sperm sample consists of: (A) the ratio between (i) the combination of the AA G_{M1} localization pattern and APM G_{M1} localization pattern and (ii) the combination of all the G_{M1} labeled localization patterns, (B) the volume of the sperm sample, and (C) a product of (a) the ratio between (i) the combination of the AA G_{M1} localization pattern and APM G_{M1} localization pattern and (ii) the combination of all the G_{M1} labeled localization patterns, and (b) the volume of the sperm sample.

In some embodiments, a classifier in the one or more pre-trained fertility classifiers is a nonlinear regression model. In some embodiments, a classifier in the one or more pre-trained fertility classifiers is a logistic regression model (e.g., of the form:

$$f ( X ) = \frac{1}{1 + exp ( - ( {β}_{0} + {\sum }_{j = 1}^{i} {β}_{j} {X}_{j} ) )}$$

wherein: ƒ(X) is a measure of fertility, i is a positive integer, α is parameter determined during training of the pre-trained classifier, β₀, β₁, . . . , βᵢ are parameters determined during training of the pre-trained classifier, and each Xⱼ in {X₁, . . . , Xᵢ} is a datum in the data obtained from the sperm sample (e.g., including one or more of a ratio between (i) a combination of the AA GM1 localization pattern and APM GM1 localization patterns and (ii) a combination of all the GM1 labeled localization patterns, a volume of the sperm sample, a concentration of sperm in the sperm sample, a motility of sperm in the sperm sample, and an interaction term thereof).

In some embodiments, the capacitating conditions included exposure of the portion of the sperm sample to one or more of bicarbonate ions, calcium ions, and a mediator of sterol efflux. In some embodiments, the mediator of sterol efflux comprises 2-hydroxy-propyl-β-cyclodextrin, methyl-β-cyclodextrin, serum albumin, high density lipoprotein, phospholipid vesicles, fetal cord serum ultrafiltrate, fatty acid binding proteins, or liposomes. In some embodiments, the mediator of sterol efflux comprises 2-hydroxy-propyl-β-cyclodextrin.

In some embodiments, the fixative comprises paraformaldehyde, glutaraldehyde or a combination thereof.

In some embodiments, the labeling molecule for G_{M1} localization patterns comprises a fluorescently-labeled cholera toxin b subunit.

In some embodiments, the identifying step was performed from 2 to 24 hours after the exposing step.

In some embodiments, prior to the exposing step, the portion of the sperm sample was treated to decrease the viscosity of the portion of the sperm sample using a wide orifice pipette made of non-metallic material and using a reagent that does not damage sperm membranes.

In some embodiments, the present disclosure provides a system for training a fertility classifier for characterizing a fertility status of a male, the system comprising: at least one processor and memory addressable by the at least one processor, the memory storing at least one program for execution by the at least one processor, the at least one program comprising instructions for: A) obtaining a training set that comprises data from sperm samples from a plurality of males associated with a known outcome of an attempt at assisted reproduction (e.g., intra-uterine insemination (IUI)), wherein the data from each respective semen sample comprises a ratio between (i) a combination of the AA G_{M1} localization pattern and APM G_{M1} localization pattern and (ii) the combination of all the G_{M1} labeled localization patterns of sperm in the respective semen sample (e.g., a ratio of sperm displaying a capacitated state to a total number of assigned sperm), and B) training one or more fertility classifiers based on at least a correspondence between the outcome of the assisted reproduction attempt and the corresponding ratio between (i) a combination of the AA G_{M1} localization pattern and APM G_{M1} localization pattern and (ii) the combination of all the G_{M1} labeled localization patterns of sperm in each respective semen sample.

In some embodiments, the ratio between (i) the combination of the apical acrosome (AA) G_{M1} localization pattern and acrosomal plasma membrane (APM) G_{M1} localization pattern and (ii) the combination of all G_{M1} labeled localization patterns of sperm for each respective sperm sample from the plurality of males was determined by a method comprising: exposing, in vitro, a portion of the sperm sample from a respective male in the plurality of males to capacitating conditions, thereby forming a capacitated sperm sample, fixing the capacitated sperm sample with a fixative, thereby forming a fixed in vitro capacitated sperm sample, treating the fixed in vitro capacitated sperm sample with a labeling molecule for G_{M1} localization patterns, wherein the labeling molecule has a detectable label, thereby forming a labeled fixed in vitro capacitated sperm sample, identifying a plurality of G_{M1} labeled localization patterns for the labeled fixed in vitro capacitated sperm sample, said plurality of G_{M1} labeled localization patterns comprising an AA G_{M1} localization pattern, an APM G_{M1} localization pattern, a Lined-Cell G_{M1} localization pattern and all other labeled G_{M1} localization patterns, assigning the AA G_{M1} localization pattern and the APM G_{M1} localization pattern to a capacitated state, assigning the Lined-Cell G_{M1} localization pattern and all other labeled G_{M1} localization patterns to a non-capacitated state, and comparing (i) the combination of the AA G_{M1} localization pattern and APM G_{M1} localization pattern to (ii) the combination of all the G_{M1} labeled localization patterns of sperm.

In some embodiments, the capacitating conditions include exposure of the portion of the sperm sample to one or more of bicarbonate ions, calcium ions, and a mediator of sterol efflux. In some embodiments, the mediator of sterol efflux comprises 2-hydroxy-propyl-β-cyclodextrin, methyl-β-cyclodextrin, serum albumin, high density lipoprotein, phospholipid vesicles, fetal cord serum ultrafiltrate, fatty acid binding proteins, or liposomes. In some embodiments, the mediator of sterol efflux comprises 2-hydroxy-propyl-β-cyclodextrin.

In some embodiments, the fixative comprises paraformaldehyde, glutaraldehyde or a combination thereof.

In some embodiments, the labeling molecule for G_{M1} localization patterns comprises a fluorescently-labeled cholera toxin b subunit.

In some embodiments, the identifying step is performed from 2 to 24 hours after the exposing step.

In some embodiments, the method used to determine the ratio between (i) the combination of the AA G_{M1} localization pattern and APM G_{M1} localization pattern and (ii) the combination of all the G_{M1} labeled localization patterns of sperm for each respective semen sample further comprised, prior to the obtaining step, treating the portion of the sperm sample to decrease the viscosity of the sperm sample using a wide orifice pipette made of non-metallic material and using a reagent that does not damage sperm membranes.

In some embodiments, the data used to train the one or more fertility classifiers consists of the ratio between (i) the combination of the AA G_{M1} localization pattern and APM G_{M1} localization pattern and (ii) the combination of all of the G_{M1} labeled localization patterns from each respective semen sample from the plurality of males. In some embodiments, the data used to train the fertility classifier further comprises, from each respective semen sample from the plurality of males, one or more datum selected from the group consisting of: (A) a volume of the sperm sample, (B) a concentration of sperm in the sperm sample, (C) a motility of sperm in the sperm sample, and (D) an arithmetic combination of any two of (a) the ratio between (i) the combination of the AA G_{M1} localization pattern and APM G_{M1} localization pattern and (ii) the combination of all G_{M1} labeled localization patterns, (b) the volume of the sperm sample, (c) the concentration of sperm in the sperm sample, and (d) the motility of sperm in the sperm sample. In some embodiments, the data used to train the fertility classifier consists of, from each respective sperm sample from the plurality of males: (A) the respective ratio between (i) the combination of the AA G_{M1} localization pattern and APM G_{M1} localization pattern and (ii) the combination of all the G_{M1} labeled localization patterns, (B) the volume of the sperm sample, and (C) a product of (a) the ratio between (i) the combination of the AA G_{M1} localization pattern and APM G_{M1} localization pattern and (ii) the combination of all the G_{M1} labeled localization patterns, and (b) the volume of the sperm sample.

In some embodiments, a classifier in the one or more fertility classifiers is a nonlinear regression model. In some embodiments, a classifier in the one or more fertility classifiers is a logistic regression model, e.g., of the form:

$$f ( X ) = \frac{1}{1 + exp ( - ( {β}_{0} + {\sum }_{j = 1}^{i} {β}_{j} {X}_{j} ) )}$$

wherein: ƒ(X) is a measure of fertility, i is a positive integer, α is parameter determined during training of the pre-trained classifier, β₀, β₁, . . . , βᵢ are parameters determined during training of the pre-trained classifier, and each Xⱼ in {X₁, . . . , Xᵢ} is a datum in the data obtained from the sperm sample (e.g., including one or more of a ratio between (i) a combination of the AA GM1 localization pattern and APM GM1 localization patterns and (ii) a combination of all the GM1 labeled localization patterns, a volume of the sperm sample, a concentration of sperm in the sperm sample, a motility of sperm in the sperm sample, and an interaction term thereof).

In an embodiment of the invention, the mean Cap-Score™ for a normal, fertile male is from about 30 to about 40, or more. In an embodiment, the mean Cap-Score™ is from about 32 to about 38. In an embodiment, the mean Cap-Score™ is from about 34 to about 36. In an embodiment, the mean Cap-Score™ is about 35.3.

In an embodiment of the invention, the mean percent likelihood of pregnancy (or probability of pregnancy) is from about 35% to about 50%. In an embodiment, the mean probability of pregnancy is from about 37% to about 48%. In an embodiment of the invention, the mean probability of pregnancy is from about 39% to about 46%. In an embodiment, the mean probability of pregnancy is from about 41% to about 44%. In an embodiment of the invention, the mean probability of pregnancy is about 43%.

In an embodiment, the present disclosure provides for a method for identifying a reproductive approach for couples trying to achieve pregnancy. In an embodiment, the method comprises obtaining a Cap-Score for an individual as set forth above and running the Cap-Score through the logistical regression analysis discussed above to obtain a percent likelihood of pregnancy from the male perspective within the first three months of trying to conceive through natural conception or within 3 rounds of intrauterine insemination (IUI). This value will inform a physician and patient of the most effective and efficient course of reproductive therapy, and ultimately will save time, money, and effort for the patient, the doctor, and the insurance companies. For example, a male with a high Cap-Score (Male 1), after running the Cap-Score through the logistical regression analysis, will have a higher percent likelihood of achieving a pregnancy within the first three months of trying to conceive through natural conception or within 3 rounds of IUI than a male with a low Cap-Score (Male 2). Therefore, a physician may choose to provide fertility stimulation drugs to the partner of Male 1 and instruct them to try to conceive naturally, whereas for Male 2, the physician may recommend a more rigorous form of reproductive therapy such as, for example, in vitro fertilization, or a sperm donor.

The disclosures of US Patent Publications Nos. 2017-0184605, 2017-0234857, and 2017-0248584 are incorporated by reference herein in their entireties.

The invention is further described through the following illustrative examples, which are not to be construed as restrictive.

### Example 1

This example provides demonstration of G_{M1} localization patterns obtained with human sperm. Ejaculated sperm were collected from male donors, and allowed to liquefy for 20 mins at 37° C., and then volume, initial count, motility and morphology assessments were performed. 1 ml of the semen sample was layered on top of 1 ml of a density gradient (90% Enhance-S; Vitrolife, San Diego, Calif., USA) in a 15 ml conical tube. The tube was centrifuged at 300×g for 10 minutes. The bottom 1 ml fraction was transferred to a new 15 ml tube and then resuspended in 4 ml of mHTF. This was centrifuged at 600×g for 10 minutes. The supernatant was removed and the pellet of sperm was resuspended in 0.5 ml of mHTF. The washed sperm were then evaluated for concentration and motility. Equal volumes of sperm were then added to two tubes, such that the final volume of each tube was 300 μl, and the final concentration of sperm was 1,000,000/ml. The first tube contained mHTF (non-capacitating condition) and the second tube contained mHTF plus 2-hydroxy-propyl-β-cyclodextrin at a final concentration of 3 mM (capacitating condition). Sperm were incubated for varying lengths of time, but 3 hours was typically used. These incubations were performed at 37° C.

At the end of the incubation period, the contents of each tube were mixed gently, and 18 μl from each tube was removed and transferred to separate microcentrifuge tubes. 2 μl of 1% (weight/volume) paraformaldehyde was added to achieve a final concentration of 0.1%. In another embodiment, 0.1% (weight/volume) paraformaldehyde was added to achieve a final concentration of 0.01%. These tubes were mixed gently and incubated at room temperature for 15 minutes, at which time 0.3 μl of 1 mg/ml cholera toxin b subunit was added. The contents of the two tubes were again mixed gently and allowed to incubate for an additional 5 minutes at room temperature. From each tube, 5 μl was removed and placed on a glass slide for evaluation by fluorescence microscopy. To provide a counter-stain, speeding determination of focal planes and increasing longevity of the fluorescence signal, 3 μl of DAPI/Antifade was sometimes added.

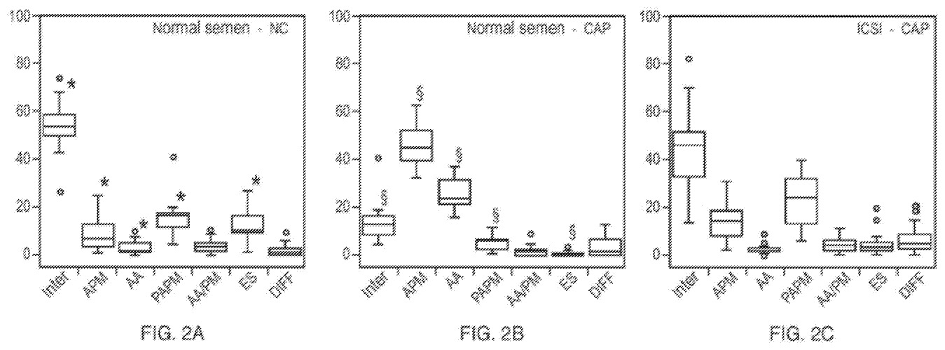

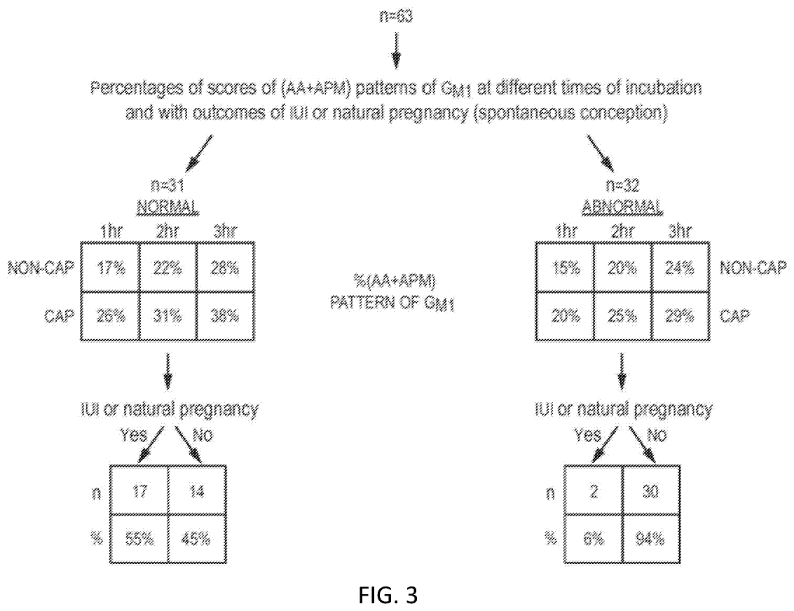

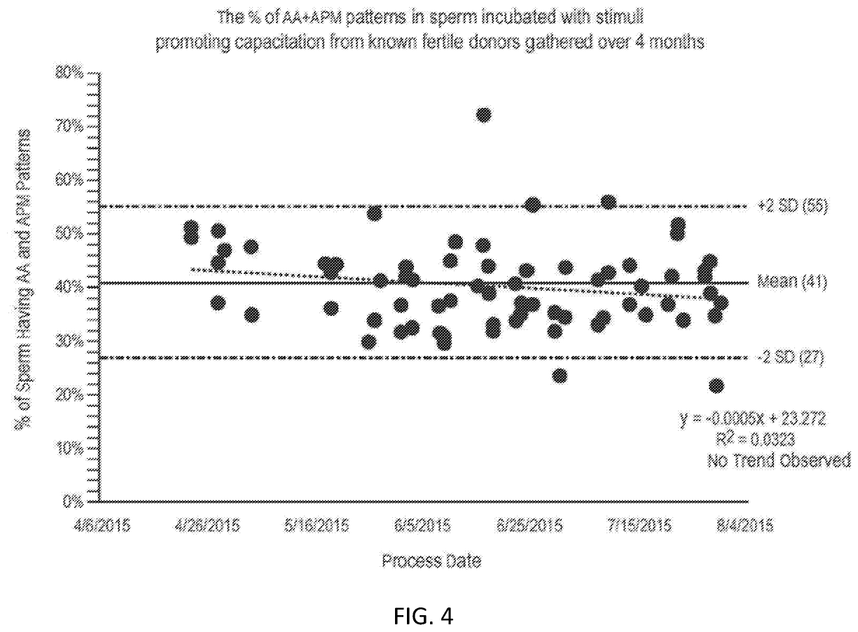

As shown in FIG. 2, localization patterns of G_{M1} in normal human sperm reflect response to capacitating conditions. Full response is seen only in men with normal fertility; the responsive pattern was largely reduced or absent in men with unexplained infertility who have failed on previous attempts at intrauterine insemination (IUI) or in vitro fertilization (IVF). FIG. 1 shows the G_{M1} patterns in human sperm. However, for the purpose of the diagnostic assay, patterns reflecting abnormalities such as PAPM, AA/PA, ES, and DIFF can be grouped for ease of analysis. FIGS. 2A-2C show the relative distributions of the different patterns in normal semen incubated under non-capacitating conditions (NC; FIG. 2A), or capacitating conditions (CAP; FIG. 2B). A reduction in INTER pattern is seen in normal semen upon exposure to CAP (FIG. 2C), while significant increases in the AA pattern and the APM pattern are also seen. In comparison with these normal data, sperm from a group of men known to have unexplained infertility were also subjected to the G_{M1} assay. In these sperm, there was almost no increase in the AA pattern or the APM pattern under capacitating conditions.

### Example 2

In this example, clinical histories of 34 patients were studied to perform a close analysis of their G_{M1} assay scores relative to history of ever achieving clinical evidence of pregnancy. A male patient was defined as “fertile” if a patient couple achieved some evidence of fertilization/clinical pregnancy (even if limited to biochemical evidence or a sac without heartbeat on ultrasound) within 3 or fewer cycles.

Analysis of the data for these 34 patients revealed that if one applied a cut-off of 40% (APM+AA) for the score of the capacitated samples at the 3-hour time point, then 7/8 who “passed” (having a score of 39.5% or greater), were found to have been designated “fertile” (87.5%). Of the 26 who “failed” (having a score of 39.4 or less), only 3/26 had evidence of clinical pregnancy (11.5%). (see Table 1 below).

If one reduces the cutoff, it would be predicted that more people who are clinically sub-fertile will get a passing score and the percentage that pass the assay and are fertile within 3 cycles should go down. Interestingly, the result was not a smooth gradient or continuous curve in terms of fertility (as defined by the ≤=3 cycle criterion). That is, whether one failed the assay as defined at 40 or 35 didn't correlate with any significant change in chance of fertility, which was always low (between 11.5-14.3%). Conversely, passing the assay at 35 vs 40 corresponded with a very large difference in chances of fertility (ranging from 53.8-87.5%, respectively). To reinforce and reiterate this point, a change in 5% of the combined APM+AA percentages corresponded with over a 30% change in history of fertility.

These results suggest that male fertility is more like a “step function,” in which ranges of scores for the male fertility assay correspond with categorizations of “fertile,” “sub-fertile” or “infertile,” rather than small changes in scores equating with correspondingly small but continuous changes in male fertility (chance of achieving clinical pregnancy). These data indicate strongly that a score of roughly 38.5-40 would be the cut-off between designations of “sub-fertile” or “fertile.” Further examination of the data suggests that a cut-off of <14.5% could be used as a designation of likely “infertile.”

| Cut-Off | Fertile Defined on Conceiving Within </= 3 cycles | |
|---|---|---|
| 39.5 | Pass | 8 (7/8 fertile = 87.5%) |
| | Fail | 26 (3/26 fertile = 11.5%) |
| 38.5 | Pass | 8 (7/8 fertile = 87.5%) |
| | Fail | 26 (3/26 fertile =11.5%) |
| 37.5 | Pass | 11 (7/11 fertile = 63.6%) |
| | Fail | 23 (3/23 fertile = 13.0%) |
| 36.5 | Pass | 11 (7/11 fertile = 63.6%) |
| | Fail | 23 (3/23 fertile = 13.0%) |
| 35.5 | Pass | 13 (7/13 fertile = 53.8%) |
| | Fail | 21 (3/21 fertile = 14.3%) |

Summarizing data for these men, who were all similar in terms of average semen parameters, suggest the following ranges (based on absolute scores): Infertile: <14.5, sub-fertile: 14.5-38.4, fertile: ≥38.5.

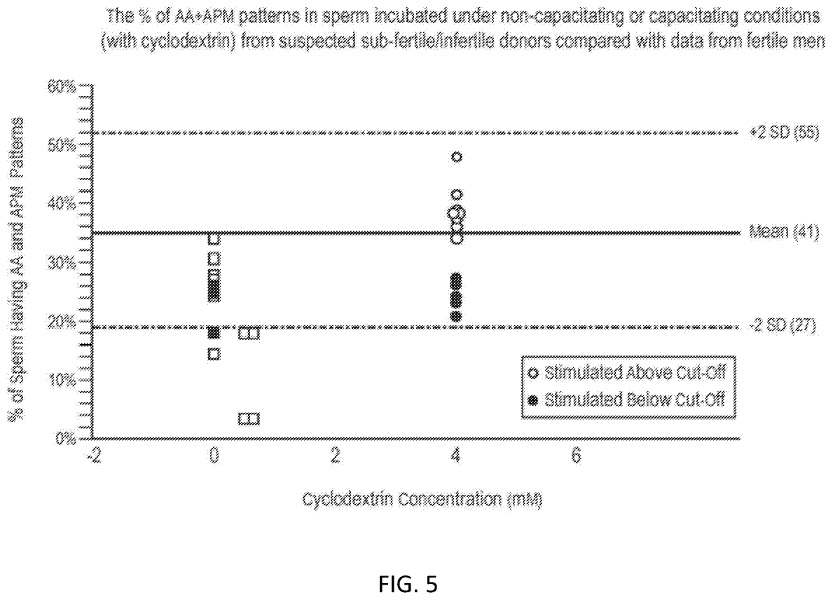

Alternatively, one can evaluate the fertility of a sample by comparing the change in relative number of the APM and/or AA patterns over the time of incubation under capacitating conditions, or against the relative number observed under non-capacitating conditions. For example, one could compare the APM+AA relative number after 3 hours of incubation in capacitating conditions with the relative number of those patterns at the start of incubation. In yet another embodiment, one might compare the change in APM and/or AA frequencies with results obtained from successive time points (such as 1, 2, and 3 hours). In effect, one can plot the relative frequencies on the Y axis and time points on the X axis, and evaluate the slope or rate of change of the increasing number of one or more of the INTER, APM and/or AA samples under non-capacitating and capacitating conditions. When this approach to the analysis was performed in a group of 63 patients, 31 men with scores matching the normal reference group were identified, with baseline G_{M1} patterns of 17%-22%-28% in non-capacitating and 26%-31%-38% in capacitating media, respectively over 1, 2, and 3 hours of incubation (see FIG. 3). 32 men with below reference values of 15%-20%-24% in non-capacitating and 20%-25%-29% in capacitating media were identified. Semen analysis parameters of number, motility and percent normal morphology (using strict WHO criteria) were comparable between the two groups. The population with normal range G_{M1} patterns had an intrauterine insemination (IUI) pregnancy rate of 45.2% (14/31) of which 8 (25.8%) generated at least one fetal heartbeat. Three additional couples in this group became pregnant on their own. For men with below-reference G_{M1} patterns, the IUI clinical pregnancy rate was only 6.3% (2/32; P=0.03). In this cohort, 13 underwent ICSI and 6 became pregnant (46.2%).

### Example 3

Sperm cells were treated as described in Example 1 but incubated in fixative for 24 hours. The labeled sperm cells were then evaluated by fluorescence microscopy. A new G_{M1} localization pattern, Lined-Cells was identified as illustrated in FIGS. 6A, 6B, 6C and 6D. In Lined-cells, as illustrated in FIG. 6A, there is G_{M1} signal at the bottom of the equatorial segment/top of the post acrosomal region, and at the plasma membrane overlying the acrosome. The signal is evenly distributed in the post acrosome/equatorial region and the plasma membrane overlying the acrosome. There is also a band at the equatorial segment that lacks signal. As illustrate in FIG. 6B, the signal at the plasma membrane overlying the acrosome is brighter than the signal at the post acrosome/equatorial band. As illustrated in FIGS. 6C and 6D, the signal found at the post acrosome/equatorial band is brighter than the signal at the plasma membrane overlying the acrosome.

Sperm cells from a single donor were washed, incubated under both capacitating (Stim) and non-capacitating (Non-Stim) conditions and then scored both on day 0 (maintained in fix for approximately 5 hours) and day 1 (maintained in fix for approximately 27.5 hours). There was little change in the percentage of Lined-cells from day 0 to day 1 for the Stim treatment. In contrast, the percent of lined cells from day 0 to day 1 increased from 3 to 22% for the Non-Stim treatment. In conjunction with this change, there was a subsequent decrease in INTER from 76 to 51%. These data are consistent with lined cell patterns developing on day 1 from cells having an inter pattern on day 0.

TABLE 1

| | % AA | % APM | % Inter | % Lined Cells | % Other |
|---|---|---|---|---|---|
| Non-Stim day 0 | 0 | 16 | 76 | 3 | 6 |
| Stim day 0 | 7 | 19 | 62 | 4 | 8 |
| Non-Stim day 1 | 4 | 14 | 51 | 22 | 10 |
| Stim day 1 | 4 | 24 | 53 | 3 | 16 |

### Example 4

Cells from a single donor were washed, incubated under both capacitating (Stim) and non-capacitating (Non-Stim) conditions and then scored both on day 0 (maintained in fix for approximately 4 hour) and day 1 (maintained in fix for approximately 25 hours). Since few lined cells were observed on day 0, similar Cap-Scores were obtained on day 0 with or without including the number of Lined-cells for determining the Cap-Scores. However, with the emergence of Lined-cells on day 1, different Cap-Score™ values could be obtained depending on how the Lined-cells were interpreted. For example, when Lined-cells were treated as Non-Capacitated, similar Cap-Scores™ were obtained for both the Stim and non-Stim treatments. However, if the Lined-cells were treated as capacitated, separated or removed from the Cap-Score™ calculation, greater Cap-Scores were obtained for the Non-Stim treatment than for the Stim treatment. Having larger Cap-Score™ values for the Non-Stim treatment makes no sense, as these sperm cells were incubated under basal conditions and thus would not have shown G_{M1} patterns associated with capacitation. These observations provide further complementary evidence that Lined-cells represent a non-capacitated state and should be treated as such when calculating Cap-Score™

TABLE 2

| | Cap-Score ™ computed with: | | | |
|---|---|---|---|---|
| | Lined cells as | | | |
| | Non- | Lined cells as | Lined cells | Lined Cells |
| | Capacitated | Capacitated | separated | removed |
| Non-Stim day 0 | 18 | 20 | 20 | 19 |
| Stim day 0 | 28 | 31 | 30 | 29 |
| Non-Stim day 1 | 35 | 52 | 52 | 42 |
| Stim day 1 | 36 | 39 | 38 | 37 |

### Example 5

Forty different semen samples, from 18 unique donors were washed, the sperm were incubated under both capacitating (Stim) and non-capacitating (Non-Stim) conditions and then scored both on day 0 and day 1. A significant correlation is observed between Stim Cap-Score™ values obtained on day 0 and day 1 when Lined-cells are treated as non-capacitated (r=0.32; n=40; p<0.05). In contrast, no correlation is observed between day 0 and day 1 when Lined-cells are treated as capacitated, separated into either capacitated or non-capacitated bins based on G_{M1} localization pattern, or simply removed from the Cap-Score™. These observations support the view that the Lined-cells localization pattern develops as sperm are maintained overnight and that on day 0 these sperm exhibit an inter/non-capacitated pattern. The treatment of Lined-cells as non-capacitated, stabilizes the Cap-Score™ over time and is consistent with this pattern reflecting cells that are infertile. What's more, these data demonstrate that interpretation of the Lined-cells as non-capacitated is applicable to the population. Nonetheless, the appearance of Lined-cells on day 1 is donor dependent. This raises the possibility that observation of these Lined-cells may provide additional information about these donors and the ability of their sperm to fertilize.

### Example 6

Data from sperm samples (i.e., semen samples) collected from 56 male subjects who used intra-uterine insemination (IUI) to try and become pregnant with their partner, at a single fertility clinic, was used as a training set for various fertility pattern recognition classifiers. Each data entry included whether the couple became pregnant using IUI, a Cap-Score™, a volume of the sperm sample, a concentration of sperm in the sperm sample, and a motility of sperm in the sperm sample.

In a first attempt logistic regression was used to build a classifier for characterizing the fertility of the male. Logistic regression is a technique that models categorical data by assuming that the probabilities of the categories are determined by a transformation of a linear model on a set of given variables. For binary data such as a pregnant/not pregnant dichotomy, what is assumed to be linear in the variables is the logit of the success probabilities:

$$log it ( p ) = log ( \frac{p}{1 - p} ) = A x$$

where x is some vector of variables, and A is a set of coefficients, one for each variable. The four variables were Cap-Score, motility, concentration, and volume. For linear discriminant analysis (LDA), these variables were centered and scaled, but the models used the original values. Models using all combinations of the four variables (15 models), and interaction models selected through stepwise variable selection. The two best models were one based on Cap-Score alone, and another that used Cap-Score and volume. Cap-Score alone was predictive of pregnancy outcome (p=0.05; probability of generating pregnancy (PGP) range: 6.97-58.7%).

One measure of how well the model explains the data, deviance, suggests that the more complex model (e.g., using Cap-Score and volume) is a slightly better model than the model based on Cap-Score alone. However, more complex models tend to have lower deviance just because they are more flexible.

AIC takes model complexity into account in order to assess whether the more complex model is actually more informative or whether it only appears more informative because it is more flexible. AIC suggests the model using Cap-Score alone is slightly better than the complex model (e.g., using Cap-Score, volume, and an interaction term that is the product of the two).

The deviance and AIC measures do not clearly favor one model over the other. However, we using the model based on Cap-Score alone is advantageous for at least three reasons. First, the model based on Cap-Score alone is simpler, and simpler models tend to be more robust. Second, volume is problematic on medical grounds. For moderate values, increased volume is associated with increased fertility, but large values are associated with decreased fertility. Third, the output of the logistic model is directly interpretable as the probability of success, as discussed further below.

Statistical measures for all fifteen combinations of the variables (excluding interaction terms) are shown in Table 3, below. As described above, the model using Cap-Score alone was associated with a lower AIC than any other model, including the more complex models associated with lower deviances.

TABLE 3
Statistical measures of fit for logistic regression models
of male fertility from a single IUI clinic.

| | Single Clinic | |
|---|---|---|
| Model | Deviance | AIC |
| Cap-Score | 61.71 | 65.71 |
| Motility | 63.50 | 67.50 |
| Concentration | 64.45 | 68.45 |
| Volume | 65.17 | 69.17 |
| Cap-Score + Motility | 60.59 | 66.59 |
| Cap-Score + Concentration | 60.45 | 66.45 |
| Cap-Score + Volume | 60.06 | 66.06 |
| Motility + Concentration | 63.24 | 69.24 |
| Motility + Volume | 62.76 | 68.76 |
| Concentration + Volume | 64.29 | 70.29 |
| Cap-Score + Motility + Concentration | 60.07 | 68.07 |
| Cap-Score + Motility + Volume | 58.82 | 66.82 |
| Cap-Score + Concentration + Volume | 59.46 | 67.46 |
| Concentration + Motility + Volume | 62.72 | 70.72 |
| Cap-Score + Concentration + Motility + Volume | 58.76 | 68.76 |

The LDA model, described above, has been used to provide a simple subdivision of male fertility into sets of high, medium, and low probability of pregnancy, with the thresholds set by computing the two empirical cumulative distribution functions (ECDFs) of the Pregnant (P) and Not Pregnant (NP) and finding the points of maximum difference. An important advantage to the logistic regression model over the LDA model is that the logistic model estimates an individual's probability of conception rather than providing a triage category. The logistic model could be used to create triage categories as well, but it is more informative than that. With LDA there is no straightforward interpretation of individual values of the discriminant.
The triage thresholds are the result of a statistical procedure, and if a different sample had occurred, the intervals defined by those thresholds would have different percentages of the population. The uncertainties of those percentages were calculated by using the bootstrap, which samples with replacement from the original population to simulate the effect of a new population. We discovered that the percentages for the thresholds have a standard deviation of about 9%. That is, we can only say with confidence that a percentage given as 39% is between 21% and 57%. This is true for the triage percentages calculated from either the LDA analysis or the logistic regression.
The individual probabilities referred to above also have uncertainties that can be evaluated by probabilistic simulation, and their variability is of the same order of magnitude. With the current data, no figure coming out of either analysis is guaranteed accurate beyond the first decimal place.
Other models based on machine learning were also considered, however, they were not as successful as the logistic regression approach outlined above. For example, an attempt to build a neural network based on the four variables measured in the single facility study described above (e.g., Cap-Score™, volume of the sperm sample, concentration of sperm in the sperm sample, and motility of sperm in the sperm sample) failed to converge. Likewise, an unsupervised method called k-means clustering called the central majority of the data in one cluster and broke off tiny clusters around the edges.

Two other statistical methods did successfully run, random forests and nearest neighbor classification. However, these methods worked poorly compared to the logistic regression model. The random forest model, trained on the single clinic data, classified 58.8% of the non-pregnant cases correctly, but classified only 22.7% of the pregnant cases correctly. Nearest neighbor classification identified 59% of the non-pregnant cases correctly, but classified only 35.3% of the pregnant cases correctly. The two techniques generate inferior predictions, principally because they are local techniques that cannot “see” the entire body of the data at once. The two methods are quite different, but produced very similar results: only slightly better than guessing for most cases. Larger data sets would at least better determine the precision of logistic regression or linear discriminant analysis; with these techniques there is no reason to expect that training on more samples would cause them to perform better.

It was also considered whether the models described above could be used to predict 17 cases of natural pregnancies, which were excluded from the training set. Using the LDA model, only 7 of the 17 cases scored in the “High” category defined by that analysis, while 4 scored “Medium” and 6 scored “Low”. With the logistic regression model, the median prediction of pregnancy was just 34%, only three cases had predictions above 50%, and the highest prediction was 56%. It is clear that the natural pregnancy data does not “look like” the IUI pregnancy data.

### Example 7

To determine whether a single logistic regression model could fit data taken at multiple fertility clinics, data from sperm samples (i.e., semen samples) from 124 male subjects who used intra-uterine insemination (IUI) across five different clinics. Each data entry included whether the couple became pregnant using IUI, a Cap-Score, a volume of the sperm sample, a concentration of sperm in the sperm sample, and a motility of sperm in the sperm sample. Logistic regression models were calculated, as in Example 5, for all combinations of the four variables (15 models), and interaction models selected through stepwise variable selection. Statistical measures for all fifteen combinations of the variables (excluding interaction terms) are shown in Table 4, below.

TABLE 4
Statistical measures of fit for logistic regression models of
male fertility from five IUI clinics.

| | Multiple Clinic | |
|---|---|---|
| Model | Deviance | AIC |
| Cap-Score | 148.56 | 152.56 |
| Motility | 157.67 | 161.67 |
| Concentration | 158.54 | 162.54 |
| Volume | 159.41 | 163.41 |
| Cap-Score + Motility | 147.47 | 153.47 |
| Cap-Score + Concentration | 147.75 | 153.75 |
| Cap-Score + Volume | 148.55 | 154.55 |
| Motility + Concentration | 157.47 | 163.47 |
| Motility + Volume | 157.57 | 163.57 |
| Concentration + Volume | 158.19 | 164.19 |
| Cap-Score + Motility + Concentration | 147.22 | 155.22 |
| Cap-Score + Motility + Volume | 147.46 | 155.46 |
| Cap-Score + Concentration + Volume | 147.72 | 155.72 |
| Concentration + Motility + Volume | 157.25 | 165.25 |
| Cap-Score + Concentration + Motility + Volume | 147.21 | 157.21 |

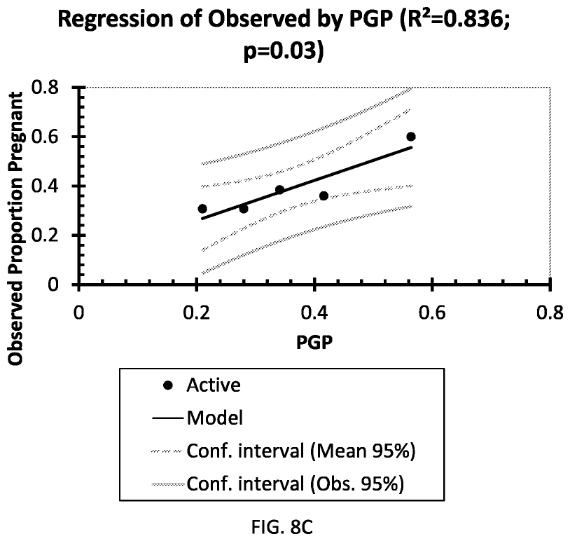

As reported for the single-clinic data, the logistic regression model using Cap-Score alone (represented in FIG. 7) was associated with a lower AIC than any other model. Cap-Score alone was predictive of pregnancy outcome (p<0.001, PGP range 6.97-80.7%). Incorporation of data from multiple sites resulted in a very slight drop in the quality measures of the model, perhaps indicating some non-uniformity in the methods used among the different facilities. However, the resulting model is adequate to describe any of them and has reduced uncertainty as compared to models calculated based on only the single clinic data from any of the five clinics. The bootstrapping exercise described in Example 6 was repeated for the multi-clinic data to determine the uncertainties of the resulting probability predictions. It was found that the uncertainties dropped from the previous standard deviation of about 9% (in the single clinic study) down to 4% (for the multi-clinic study). As predicted, the additional data reduces the error of prediction estimates.

### Example 8

Semen analysis fails to diagnose many cases of male factor fertility because it lacks a functional test that provides information regarding the ability of sperm to fertilize, and focuses only on more descriptive characteristics of sperm and semen such as concentration, motility, morphology, and volume. The Cap-Score™ has previously been shown to have a strong correlation with male fertility using low and normal fertility “cut off” points. However, male fertility is a continuum, and the inventors have intended to show, using logistic regression, how Cap-Score™ relates to the probability of generating a pregnancy. Here, the relationship between the predicted probability of generating a pregnancy and actual IUI outcomes was tested.

Cap-Scores™ and outcomes for 292 male subjects were obtained from six clinics. Of the 292 subjects, 128 completed treatment (i.e., became pregnant through intrauterine insemination (IUI) within 3 cycles or completed 3 attempts without generating a pregnancy). The PGP model was tested in two ways. Test 1: The new outcomes were added to the prior 124 outcomes of Example 7 and the model was re-run to determine change. Test 2: The 128 outcomes were divided into rank-ordered groups of roughly equal size and the proportion of individuals successfully generating a pregnancy within each group was compared to the average predicted PGP within that group using a linear regression approach as described herein.

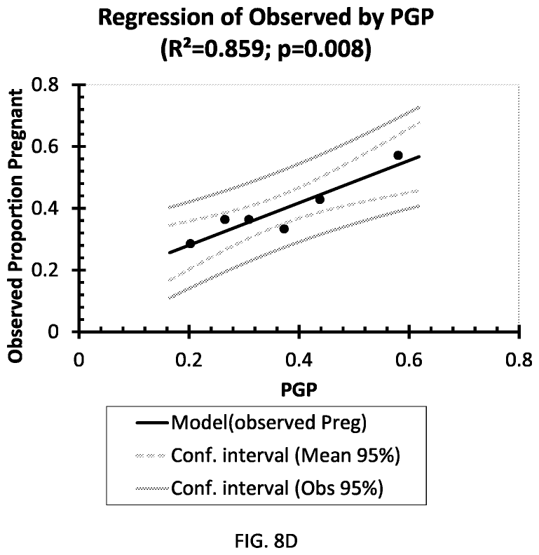

Test 1 results. Only a slight average change was observed when the 128 new data points were added to the previous 124 data points from Example 7 and a new logistic regression model was generated. The majority of the change derived from the ends of the curve where there were the fewest data points. The regression equation for the 124 data points was PGP=1/[1+exp[−[−2.86+0.08*Cap-Score™]]], with a p-value of p<0.01, and the regression equation from the 252 data points (124+128) was PGP=1/[1+exp[−[−2.26+0.06*Cap-Score™]]], with a p-value of P<0.001. FIGS. 8A and 8B illustrate the relationship between Cap-Score™ and PGP for the n=124 group and n=252 group, respectively. For both groups, a strong association between Cap-Score™ and PGP was observed using the logistic regression model disclosed herein, and, as shown in FIG. 8B, with more data points, the fit of the model improved.

Test 2 results. The 128 outcomes were divided into 5 groups and the PGPs calculated for each group; the outcomes were also divided into 6 groups and the PGPs calculated for each group. When predicted PGPs were compared to observed pregnancies, significant linear relationships were seen for the different groups. For the n=5 group, y=0.81x+0.10; R²=0.84; p=0.03, and for the n=6 group, y=0.69x+0.14; R²=0.86; p<0.01. The slopes were not significantly different from 1 and intercepts were not significantly different from 0 (in t-tests, p>0.05). FIGS. 8C and 8D show the regression of observed pregnancies for the n=5 and n=6 groups, respectively. For both groups, the relationship shows that average PGP within a group was effectively equal to the observed proportion generating a pregnancy for that group. These results further support the demonstration of a strong association between Cap-Score™ sperm function/fertilizing ability, and the ability to generate a pregnancy.

### Example 9

Defects in sperm capacitation are highly prevalent in men having fertility exams because of questions about their fertility, even if those exams determine that the male is normospermic (i.e., that the semen analysis of volume, concentration, and motility are normal per World Health Organization (WHO) criteria). In this example, semen analysis metrics, Cap-Score™, and probability of generating a pregnancy within three cycles (PGP) were analyzed. This was a correlation study Cap-Score™, PGP, and semen analysis metrics of 1610 men were compared with results from a known fertile population of 76 men (identified as having a pregnant partner or recent father).

Semen was collected from male patients concerned with potential fertility issues. Samples were collected from 9 different clinics over a period of about 2.5 years. Semen volume, concentration, and sperm motility were assessed in the samples. A portion of the sample was fixed and sent to Androvia LifeSciences in accordance with their protocol, and analyzed for Cap-Score™ and PGP.

To assess the distribution of Cap-Scores™ and their associated PGPs in men questioning their fertility, PGPs were split into bins of ≤19%, 20-29%, 30-39%, 40-49%, 50-59%, and ≥60% and the distributions of Cap-Scores™ and PGPs were compared for the 1610 men versus the results from the population of 76 men with known fertility. Significantly more men questioning their fertility were found in the PGP bins of ≤19%, 20-29%, and 30-39%, than were expected based on the distribution in men with known fertility (p<0.001). Fifty-nine percent (59%; 948/1610) of men questioning their fertility and having semen analysis were found to be normospermic in regards to volume, concentration and motility. Of these, 65% (616/948) had a Cap-Score™ of less than or equal to 31, which is indicative of lower fertility status and lower PGP (less than 39%). Fewer men questioning their fertility had a Cap-Score™ of 32 or greater than expected from the distribution of fertile men. These results revealed that a high prevalence of men with normospermic semen metrics have reduced capacitation and/or fertilization ability in the population of men seeking fertility exams. Further, defects in sperm function were equally prevalent regardless of passing any single or multiple sperm analysis metrics; there was no difference in PGP among the different groups of patients: all patients, those having a single normal semen analysis metric (according to WHO), those having more than one normal semen analysis metric (according to WHO), or those have more than 10 million total motile cells (p=0.990). Comparatively to the fertile group, 65% of the normospermic males questioning their fertility (616/948) had a Cap-Score™ of 31 or less, with a probability of pregnancy of 39% or less, whereas only 25% (19/76) of the known fertile group had a Cap-Score™ in this range. Conversely, only 35% (322/948) of normospermic men seeking fertility exams had a Cap-Score™ of 32 or greater, with a probability of pregnancy of 40% or more, whereas 75% of the known fertile group had a Cap-Score™ in this range. These data support the conclusion that traditional semen analysis is not sufficient in identifying potential male fertility issues, and thus contributes to a high percentage of men being diagnosed with idiopathic infertility. These data support that a test of sperm capacitation (i.e., the Cap-Score analysis) would reduce the percentage of men diagnosed with idiopathic infertility, and could identify potential interventions that would improve sperm capacitation, thus improving PGP.

TABLE 5
Results of Example 9 Study.

| | | % of | % norm | % men | |
|---|---|---|---|---|---|
| | | all men | ospermic | having | |
| Cap- | | having | men having | fertility | |
| Score | PGP | fertility | fertility | exams | % fertile |
| (%) | (%) | exams | exams | >10M TMC | men |
| ≤18 | ≤19 | 8 | 6 | 7 | 1 |
| | | (133/1,610) | (58/948) | (110/1,489) | (1/76) |
| 19-25 | 20-29 | 28 | 27 | 28 | 9 |
| | | (456/1,610) | (255/948) | (412/1,489) | (7/76) |
| 26-31 | 30-39 | 32 | 32 | 32 | 14 |
| | | (513/1,610) | (303/948) | (482/1,489) | (11/76) |
| 32-36 | 40-49 | 17 | 19 | 18 | 36 |
| | | (271/1,610) | (181/948) | (262/1,489) | (27/76) |
| 37-42 | 50-59 | 9 | 10 | 9 | 24 |
| | | (144/1,610) | (93/948) | (135/1,489) | (18/76) |
| >242 | ≥60 | 6 | 6 | 6 | 16 |
| | | (93/1,610) | (58/948) | (88/1,489) | (12/76) |

Although the present disclosure has been described with respect to one or more particular embodiments, it will be understood that other embodiments of the present disclosure may be made without departing from the spirit and scope of the present disclosure, and such other embodiments are intended to be within the scope of this disclosure.

I/We claim:

1. A method comprising:
a. exposing, in vitro, a portion of a sperm sample from a male to capacitating conditions, thereby forming a capacitated sperm sample;
b. fixing the capacitated sperm sample with a fixative, thereby forming a fixed in vitro capacitated sperm sample;
c. treating the fixed in vitro capacitated sperm sample with a labeling molecule for G_{M1} localization patterns, wherein the labeling molecule has a detectable label, thereby forming a labeled fixed in vitro capacitated sperm sample;
d. identifying a plurality of G_{M1} labeled localization patterns for the labeled fixed in vitro capacitated sperm sample, said plurality of G_{M1} labeled localization patterns comprising an apical acrosome (AA) G_{M1} localization pattern, an acrosomal plasma membrane (APM) G_{M1} localization pattern, a Lined-Cell G_{M1} localization pattern and all other labeled G_{M1} localization patterns;
e. assigning the AA G_{M1} localization pattern and the APM G_{M1} localization pattern to a capacitated state;
f. assigning the Lined-Cell G_{M1} localization pattern and all other labeled G_{M1} localization patterns to a non-capacitated state; and
g. characterizing a fertility status of the male by applying one or more pre-trained fertility classifiers to data obtained from the sperm sample, wherein the data obtained from the sperm sample comprises a ratio between (i) a combination of the AA G_{M1} localization pattern and APM G_{M1} localization patterns and (ii) a combination of all the G_{M1} labeled localization patterns (e.g., a ratio of sperm displaying a capacitated state to a total number of assigned sperm).

2. The method of claim 1, wherein the data obtained from the sperm sample consists of the ratio between (i) the combination of the AA G_{M1} localization pattern and APM G_{M1} localization pattern and (ii) the combination of all the G_{M1} labeled localization patterns.

3. The method of claim 1, wherein the data obtained from the sperm sample further comprises one or more datum selected from the group consisting of
i. a volume of the sperm sample,
ii. a concentration of sperm in the sperm sample,
iii. a motility of sperm in the sperm sample, and
iv. an arithmetic combination of any two of: (a) the ratio between (i) the combination of the AA G_{M1} localization pattern and APM G_{M1} localization pattern and (ii) the combination of all the G_{M1} labeled localization patterns, (b) the volume of the sperm sample, (c) the concentration of sperm in the sperm sample, and (d) the motility of sperm in the sperm sample.

4. The method of claim 3, wherein the data obtained from the sperm sample consists of:
a. the ratio between (i) the combination of the AA G_{M1} localization pattern and APM G_{M1} localization pattern and (ii) the combination of all the G_{M1} labeled localization patterns,
b. the volume of the sperm sample, and
c. a product of (a) the ratio between (i) the combination of the AA G_{M1} localization pattern and APM G_{M1} localization pattern and (ii) the combination of all the G_{M1} labeled localization patterns, and (b) the volume of the sperm sample.

5. The method according to any one of claims 1-4, wherein a classifier in the one or more pre-trained fertility classifiers is a nonlinear regression model.

6. The method according to any one of claims 1-4, wherein a classifier in the one or more pre-trained fertility classifiers is a logistic regression model (e.g., of the form:
$$f ( X ) = \frac{1}{1 + exp ( - ( {β}_{0} + {\sum }_{j = 1}^{i} {β}_{j} {X}_{j} ) )}$$
wherein:
a. ƒ(X) is a measure of fertility,
b. i is a positive integer,
c. α is parameter determined during training of the pre-trained classifier, and
d. β₀, β₁, . . . , βᵢ are parameters determined during training of the pre-trained classifier, and
e. each Xⱼ in {X₁, . . . , Xᵢ} is a datum in the data obtained from the sperm sample).

7. The method according to any one of claims 1-6, wherein the capacitating conditions include exposure of the portion of the sperm sample to one or more of bicarbonate ions, calcium ions, and a mediator of sterol efflux.

8. The method of claim 7, wherein the mediator of sterol efflux comprises 2-hydroxy-propyl-3-cyclodextrin, methyl-β-cyclodextrin, serum albumin, high density lipoprotein, phospholipid vesicles, fetal cord serum ultrafiltrate, fatty acid binding proteins, or liposomes.

9. The method of claim 7, wherein the mediator of sterol efflux comprises 2-hydroxy-propyl-3-cyclodextrin.

10. The method according to any one of claims 1-9, wherein the fixative comprises paraformaldehyde, glutaraldehyde or a combination thereof.

11. The method according to any one of claims 1-10, wherein the labeling molecule for G_{M1} localization patterns comprises a fluorescently-labeled cholera toxin b subunit.

12. The method according to any one of claims 1-11, wherein the identifying step is performed from 2 to 24 hours after the exposing step.

13. The method according to any one of claims 1-12, further comprising the step of: prior to the exposing step, treating the portion of the sperm sample to decrease the viscosity of the portion of the sperm sample using a wide orifice pipette made of non-metallic material and using a reagent that does not damage sperm membranes.

14. A method comprising:
a. obtaining a first portion of a portion of a sperm sample from a male that has been exposed to in vitro capacitating conditions, fixed in a fixative, and stained with a labeling molecule for G_{M1} localization patterns, wherein the labeling molecule has a detectable label;
b. identifying a plurality of G_{M1} labeled localization patterns for the labeled fixed in vitro capacitated sperm sample, said plurality of G_{M1} localization patterns comprising an apical acrosome (AA) G_{M1} localization pattern, an acrosomal plasma membrane (APM) G_{M1} localization pattern, a Lined-Cell G_{M1} localization pattern and all other labeled G_{M1} localization patterns;
c. assigning the AA G_{M1} localization pattern and the APM G_{M1} localization pattern to a capacitated state;
d. assigning the Lined-Cell G_{M1} localization pattern and all other labeled G_{M1} localization patterns to a non-capacitated state; and
e. characterizing a fertility status of the male by applying one or more pre-trained fertility classifiers to data obtained from the sperm sample, wherein the data obtained from the sperm sample comprises a ratio between (i) a combination of the AA G_{M1} localization pattern and APM G_{M1} localization pattern and (ii) a combination of the G_{M1} labeled localization patterns (e.g., a ratio of sperm displaying a capacitated state to a total number of assigned sperm).

15. The method of claim 14, wherein the data obtained from the sperm sample consists of the ratio between (i) the combination of the AA G_{M1} localization pattern and APM G_{M1} localization pattern and (ii) the combination of all the G_{M1} labeled localization patterns.

16. The method of claim 14, wherein the data obtained from the sperm sample further comprises one or more datum selected from the group consisting of:
a. a volume of the sperm sample,
b. a concentration of sperm in the sperm sample,
c. a motility of sperm in the sperm sample, and
d. an arithmetic combination of any two of (a) the ratio between (i) the combination of the AA G_{M1} localization pattern and APM G_{M1} localization pattern and (ii) the combination of all the G_{M1} labeled localization patterns, (b) the volume of the sperm sample, (c) the concentration of sperm in the sperm sample, and (d) the motility of sperm in the sperm sample.

17. The method of claim 16, wherein the data obtained from the sperm sample consists of:
a. the ratio between (i) the combination of the AA G_{M1} localization pattern and APM G_{M1} localization pattern and (ii) the combination of all the G_{M1} labeled localization patterns,
b. the volume of the sperm sample, and
c. a product of (a) the ratio between (i) the combination of the AA G_{M1} localization pattern and APM G_{M1} localization pattern and (ii) the combination of all the G_{M1} labeled localization patterns, and (b) the volume of the sperm sample.

18. The method according to any one of claims 14-17, wherein a classifier in the one or more pre-trained fertility classifiers is a nonlinear regression model.

19. The method according to any one of claims 14-17, wherein a classifier in the one or more pre-trained fertility classifiers is a logistic regression model (e.g., of the form:
$$f ( X ) = \frac{1}{1 + exp ( - ( {β}_{0} + {\sum }_{j = 1}^{i} {β}_{j} {X}_{j} ) )}$$
wherein:
a. ƒ(X) is a measure of fertility,
b. i is a positive integer,
c. α is parameter determined during training of the pre-trained classifier, and
d. β₀, β₁, . . . , βᵢ are parameters determined during training of the pre-trained classifier, and
e. each Xⱼ in {X₁, . . . , Xᵢ} is a datum in the data obtained from the sperm sample).

20. The method according to any one of claims 14-19, wherein the capacitating conditions include exposure of the portion of the sperm sample to one or more of bicarbonate ions, calcium ions, and a mediator of sterol efflux.

21. The method of claim 20, wherein the mediator of sterol efflux comprises 2-hydroxy-propyl-β-cyclodextrin, methyl-β-cyclodextrin, serum albumin, high density lipoprotein, phospholipid vesicles, fetal cord serum ultrafiltrate, fatty acid binding proteins, or liposomes.

22. The method of claim 20, wherein the mediator of sterol efflux comprises 2-hydroxy-propyl-β-cyclodextrin.

23. The method according to any one of claims 14-22, wherein the fixative comprises paraformaldehyde, glutaraldehyde or a combination thereof.

24. The method according to any one of claims 14-23, wherein the labeling molecule for G_{M1} localization patterns comprises a fluorescently-labeled cholera toxin b subunit.

25. The method according to any one of claims 14-24, wherein the identifying step is performed from 2 to 24 hours after the exposing step.

26. The method according to any one of claims 14-25, further comprising the step of: prior to the obtaining step, treating the portion of the sperm sample to decrease the viscosity of the portion of the sperm sample using a wide orifice pipette made of non-metallic material and using a reagent that does not damage sperm membranes.

27. A method comprising the steps of:
a. obtaining a sperm sample, wherein at least a portion of the sperm sample has been exposed to in vitro capacitating conditions to obtain an in vitro capacitated sperm, has been exposed to a fixative, and has been stained for G_{M1}, thereby forming a labeled fixed in vitro capacitated sperm sample;
b. determining a Cap-Score of the labeled fixed in vitro capacitated sperm sample based on one or more G_{M1} labeled localization patterns, said G_{M1} labeled localization patterns being an apical acrosome (AA) G_{M1} localization pattern, a post-acrosomal plasma membrane (APM) G_{M1} localization pattern, a Lined-Cell G_{M1} localization pattern and all other labeled G_{M1} localization patterns; and
c. characterizing a fertility status of the male by applying one or more pre-trained fertility classifiers to data obtained from the sperm sample, wherein the data comprises a ratio between (i) a combination of the AA G_{M1} localization pattern and the APM G_{M1} localization pattern and (ii) a combination of all the G_{M1} labeled localization patterns (e.g., a ratio of sperm displaying a capacitated state to a total number of assigned sperm).

28. The method of claim 27, wherein the data obtained from the sperm sample consists of the ratio between (i) the combination of the AA G_{M1} localization pattern and APM G_{M1} localization pattern and (ii) the combination of all the G_{M1} labeled localization patterns.

29. The method of claim 27, wherein the data obtained from the sperm sample further comprises one or more datum selected from the group consisting of:
a. a volume of the sperm sample,
b. a concentration of sperm in the sperm sample,
c. a motility of sperm in the sperm sample, and
d. an arithmetic combination of any two of (a) the ratio between (i) the combination of the AA G_{M1} localization pattern and APM G_{M1} localization pattern and (ii) the combination of all the G_{M1} labeled localization patterns, (b) the volume of the sperm sample, (c) the concentration of sperm in the sperm sample, and (d) the motility of sperm in the sperm sample.

30. The method of claim 29, wherein the data obtained from the sperm sample consists of:
a. the ratio between (i) the combination of the AA G_{M1} localization pattern and APM G_{M1} localization pattern and (ii) the combination of all the G_{M1} labeled localization patterns,
b. the volume of the sperm sample, and
c. a product of (a) the ratio between (i) the combination of the AA G_{M1} localization pattern and APM G_{M1} localization pattern and (ii) the combination of all the G_{M1} labeled localization patterns, and (b) the volume of the sperm sample.

31. The method according to any one of claims 27-30, wherein a classifier in the one or more pre-trained fertility classifiers is a nonlinear regression model.

32. The method according to any one of claims 27-30, wherein a classifier in the one or more pre-trained fertility classifiers is a logistic regression model (e.g., of the form:
$$f ( X ) = \frac{1}{1 + exp ( - ( {β}_{0} + {\sum }_{j = 1}^{i} {β}_{j} {X}_{j} ) )}$$
wherein:
v. ƒ(X) is a measure of fertility,
vi. i is a positive integer,
vii. α is parameter determined during training of the pre-trained classifier, and
viii. β₀, β₁, . . . , βᵢ are parameters determined during training of the pre-trained classifier, and
ix. each Xⱼ in {X₁, . . . , Xᵢ} is a datum in the data obtained from the sperm sample).

33. The method according to any one of claims 27-32, further comprising the step of: prior to the obtaining step, treating the portion of the sperm sample to decrease the viscosity of the portion of the sperm sample using a wide orifice pipette made of non-metallic material and using a reagent that does not damage sperm membranes.

34. A method, comprising:
a. characterizing a fertility status of a male by applying one or more pre-trained fertility classifiers to data obtained from a sperm sample from the male, wherein the data comprises a ratio between (i) a combination of apical acrosome (AA) G_{M1} localization patterns and acrosomal plasma membrane (APM) G_{M1} localization patterns and (ii) a combination all G_{M1} labeled localization patterns in a treated portion of the sperm sample,
b. wherein the ratio between (i) the combination of the AA GM1 localization patterns and APM GM1 localization patterns and (ii) the combination of all G_{M1} labeled localization patterns is determined by:
a. exposing, in vitro, a portion of the sperm sample from the male to capacitating conditions, thereby forming a capacitated sperm sample;
b. fixing the capacitated sperm sample with a fixative, thereby forming a fixed in vitro capacitated sperm sample;
c. treating the fixed in vitro capacitated sperm sample with a labeling molecule for G_{M1} localization patterns, wherein the labeling molecule has a detectable label, thereby forming a labeled fixed in vitro capacitated sperm sample;
d. identifying a plurality of G_{M1} labeled localization patterns for the labeled fixed in vitro capacitated sperm sample, said plurality of G_{M1} labeled localization patterns comprising an AA G_{M1} localization pattern, an APM G_{M1} localization pattern, a Lined-Cell G_{M1} localization pattern and all other labeled G_{M1} localization patterns;
e. assigning the AA G_{M1} localization pattern and the APM G_{M1} localization pattern to a capacitated state;
f. assigning the Lined-Cell G_{M1} localization pattern and all other labeled G_{M1} localization patterns to a non-capacitated state; and
g. comparing (i) the combination of the AA G_{M1} localization pattern and APM G_{M1} localization pattern to (ii) the combination of all the G_{M1} labeled localization patterns.

35. The method of claim 34, wherein the data obtained from the sperm sample consists of the ratio between (i) the combination of the AA G_{M1} localization pattern and APM G_{M1} localization pattern and (ii) the combination of all the G_{M1} labeled localization patterns.

36. The method of claim 34, wherein the data obtained from the sperm sample further comprises one or more datum selected from the group consisting of:
a. a volume of the sperm sample,
b. a concentration of sperm in the sperm sample,
c. a motility of sperm in the sperm sample, and
d. an arithmetic combination of any two of (a) the ratio between (i) the combination of the AA G_{M1} localization pattern and APM G_{M1} localization pattern and (ii) the combination of all the G_{M1} labeled localization patterns, (b) the volume of the sperm sample, (c) the concentration of sperm in the sperm sample, and (d) the motility of sperm in the sperm sample.

37. The method of claim 36, wherein the data obtained from the sperm sample consists of:
a. the ratio between (i) the combination of the AA G_{M1} localization pattern and APM G_{M1} localization pattern and (ii) the combination of all the G_{M1} labeled localization patterns,
b. the volume of the sperm sample, and
c. a product of (a) the ratio between (i) the combination of the AA G_{M1} localization pattern and APM G_{M1} localization pattern and (ii) the combination of all the G_{M1} labeled localization patterns, and (b) the volume of the sperm sample.

38. The method according to any one of claims 34-37, wherein a classifier in the one or more pre-trained fertility classifiers is a nonlinear regression model.

39. The method according to any one of claims 34-37, wherein a classifier in the one or more pre-trained fertility classifiers is a logistic regression model (e.g., of the form:
$$f ( X ) = \frac{1}{1 + exp ( - ( {β}_{0} + {\sum }_{j = 1}^{i} {β}_{j} {X}_{j} ) )}$$
wherein:
a. ƒ(X) is a measure of fertility,
b. i is a positive integer,
c. α is parameter determined during training of the pre-trained classifier, and
d. β₀, β₁, . . . , βᵢ are parameters determined during training of the pre-trained classifier, and
e. each Xⱼ in {X₁, . . . , Xᵢ} is a datum in the data obtained from the sperm sample).

40. The method according to any one of claims 34-39, wherein the capacitating conditions included exposure of the portion of the sperm sample to one or more of bicarbonate ions, calcium ions, and a mediator of sterol efflux.

41. The method of claim 40, wherein the mediator of sterol efflux comprises 2-hydroxy-propyl-β-cyclodextrin, methyl-β-cyclodextrin, serum albumin, high density lipoprotein, phospholipid vesicles, fetal cord serum ultrafiltrate, fatty acid binding proteins, or liposomes.

42. The method of claim 40, wherein the mediator of sterol efflux comprises 2-hydroxy-propyl-β-cyclodextrin.

43. The method according to any one of claims 34-42, wherein the fixative comprises paraformaldehyde, glutaraldehyde or a combination thereof.

44. The method according to any one of claims 34-43, wherein the labeling molecule for G_{M1} localization patterns comprises a fluorescently-labeled cholera toxin b subunit.

45. The method according to any one of claims 34-44, wherein the identifying step was performed from 2 to 24 hours after the exposing step.

46. The method according to any one of claims 34-45, wherein, prior to the exposing step, the portion of the sperm sample was treated to decrease the viscosity of the portion of the sperm sample using a wide orifice pipette made of non-metallic material and using a reagent that does not damage sperm membranes.

47. A system for training a fertility classifier for characterizing a fertility status of a male, the system comprising:
a. at least one processor and memory addressable by the at least one processor, the memory storing at least one program for execution by the at least one processor, the at least one program comprising instructions for:
i. obtaining a training set that comprises data from sperm samples from a plurality of males associated with a known outcome of an attempt at assisted reproduction (e.g., intra-uterine insemination (IUI)), wherein the data from each respective semen sample comprises a ratio between (x) a combination of the AA G_{M1} localization pattern and APM G_{M1} localization pattern and (y) the combination of all the G_{M1} labeled localization patterns of sperm in the respective semen sample (e.g., a ratio of sperm displaying a capacitated state to a total number of assigned sperm); and
ii. training one or more fertility classifiers based on at least a correspondence between the outcome of the assisted reproduction attempt and the corresponding ratio between (i) a combination of the AA G_{M1} localization pattern and APM G_{M1} localization pattern and (ii) the combination of all the G_{M1} labeled localization patterns of sperm in each respective semen sample.

48. The system of claim 47, wherein the ratio between (i) the combination of the apical acrosome (AA) G_{M1} localization pattern and acrosomal plasma membrane (APM) G_{M1} localization pattern and (ii) the combination of all G_{M1} labeled localization patterns of sperm for each respective sperm sample from the plurality of males was determined by a method comprising:
a. exposing, in vitro, a portion of the sperm sample from a respective male in the plurality of males to capacitating conditions, thereby forming a capacitated sperm sample;
b. fixing the capacitated sperm sample with a fixative, thereby forming a fixed in vitro capacitated sperm sample;
c. treating the fixed in vitro capacitated sperm sample with a labeling molecule for G_{M1} localization patterns, wherein the labeling molecule has a detectable label, thereby forming a labeled fixed in vitro capacitated sperm sample;
d. identifying a plurality of G_{M1} labeled localization patterns for the labeled fixed in vitro capacitated sperm sample, said plurality of G_{M1} labeled localization patterns comprising an AA G_{M1} localization pattern, an APM G_{M1} localization pattern, a Lined-Cell G_{M1} localization pattern and all other labeled G_{M1} localization patterns;
e. assigning the AA G_{M1} localization pattern and the APM G_{M1} localization pattern to a capacitated state
f. assigning the Lined-Cell G_{M1} localization pattern and all other labeled G_{M1} localization patterns to a non-capacitated state; and
g. comparing (i) the combination of the AA G_{M1} localization pattern and APM G_{M1} localization pattern to (ii) the combination of all the G_{M1} labeled localization patterns of sperm.

49. The system of claim 48, wherein the capacitating conditions include exposure of the portion of the sperm sample to one or more of bicarbonate ions, calcium ions, and a mediator of sterol efflux.

50. The system of claim 49, wherein the mediator of sterol efflux comprises 2-hydroxy-propyl-β-cyclodextrin, methyl-β-cyclodextrin, serum albumin, high density lipoprotein, phospholipid vesicles, fetal cord serum ultrafiltrate, fatty acid binding proteins, or liposomes.

51. The system of claim 49, wherein the mediator of sterol efflux comprises 2-hydroxy-propyl-β-cyclodextrin.

52. The system according to any one of claims 48-51, wherein the fixative comprises paraformaldehyde, glutaraldehyde or a combination thereof.

53. The system according to any one of claims 48-52, wherein the labeling molecule for G_{M1} localization patterns comprises a fluorescently-labeled cholera toxin b subunit.

54. The system according to any one of claims 48-53, wherein the identifying step is performed from 2 to 24 hours after the exposing step.

55. The system according to any one of claims 48-54, wherein the method used to determine the ratio between (i) the combination of the AA G_{M1} localization pattern and APM G_{M1} localization pattern and (ii) the combination of all the G_{M1} labeled localization patterns of sperm for each respective semen sample further comprised, prior to the obtaining step, treating the portion of the sperm sample to decrease the viscosity of the sperm sample using a wide orifice pipette made of non-metallic material and using a reagent that does not damage sperm membranes.

56. The system according to any one of claims 47-55, wherein the data used to train the one or more fertility classifiers consists of the ratio between (i) the combination of the AA G_{M1} localization pattern and APM G_{M1} localization pattern and (ii) the combination of all of the G_{M1} labeled localization patterns from each respective semen sample from the plurality of males.

57. The system according to any one of claims 47-55, wherein the data used to train the fertility classifier further comprises, from each respective semen sample from the plurality of males, one or more datum selected from the group consisting of:
a. a volume of the sperm sample,
b. a concentration of sperm in the sperm sample,
c. a motility of sperm in the sperm sample, and
d. an arithmetic combination of any two of (a) the ratio between (i) the combination of the AA G_{M1} localization pattern and APM G_{M1} localization pattern and (ii) the combination of all G_{M1} labeled localization patterns, (b) the volume of the sperm sample, (c) the concentration of sperm in the sperm sample, and (d) the motility of sperm in the sperm sample.

58. The system of claim 57, wherein the data used to train the fertility classifier consists of, from each respective sperm sample from the plurality of males:
a. the respective ratio between (i) the combination of the AA G_{M1} localization pattern and APM G_{M1} localization pattern and (ii) the combination of all the G_{M1} labeled localization patterns,
b. the volume of the sperm sample, and
c. a product of (a) the ratio between (i) the combination of the AA G_{M1} localization pattern and APM G_{M1} localization pattern and (ii) the combination of all the G_{M1} labeled localization patterns, and (b) the volume of the sperm sample.

59. The system according to any one of claims 47-58, wherein a classifier in the one or more fertility classifiers is a nonlinear regression model.

60. The system according to any one of claims 47-58, wherein a classifier in the one or more fertility classifiers is a logistic regression model (e.g., of the form:
$$f ( X ) = \frac{1}{1 + exp ( - ( {β}_{0} + {\sum }_{j = 1}^{i} {β}_{j} {X}_{j} ) )}$$
wherein:
a. ƒ(X) is a measure of fertility,
b. i is a positive integer,
c. α is parameter determined during training of the pre-trained classifier, and
d. β₀, β₁, . . . , βᵢ are parameters determined during training of the pre-trained classifier, and
e. each Xⱼ in {X₁, . . . , X₁} is a datum in the data obtained from the sperm sample).

61. A method for identifying a reproductive approach comprising:
a. determining a percent likelihood of pregnancy using the method of claim 6 and
b. determining an appropriate reproductive approach based on the value identified in step a.
